(19) United States
(12) Patent Application Publication
(10) Pub. No.: US 2006/0019997 A1
(43) Pub. Date: Jan. 26, 2006

(54) Triazole compounds and their use as metabotropic glutamate receptor antagonists

(75) Inventors: Louise Edwards, Mississauga (CA); Methvin Isaac, Mississauga (CA); Martin Johansson, Sodertalje (SE); Johan Malmberg, Sodertalje (SE); Alexander Minidis, Sodertalje (SE); Karin Staaf, Sodertalje (SE); Abdelmalik Slassi, Mississauga (CA); David Wensbo, Sodertalje (SE); Tomislav Stefanac, Mississauga (CA); Tao Xin, Mississauga (CA)

Correspondence Address: FOLEY AND LARDNER LLP;SUITE 500, 3000 K STREET NW, WASHINGTON, DC, 20007, US

(73) Assignees: AstraZeneca AB; NPS Pharmaceuticals Inc.

(21) Appl. No.: 11/060,366

(22) Filed: Feb. 18, 2005

Related U.S. Application Data
(60) Provisional application No. 60/545,289, filed on Feb. 18, 2004.

(51) Int. Cl. A61K 31/4439; A61K 31/4196; A61K 31/4245; C07D 413/14; C07D 403/14

(52) U.S. Cl. 514/341; 514/364; 514/383; 514/384; 514/359; 546/269.1; 548/266.2; 548/255; 548/263.2

(57) ABSTRACT

The present invention relates to new compounds of formula I, 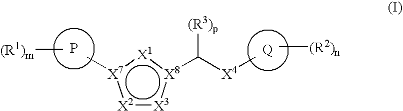
wherein P, Q, X¹, X², X³, X⁴ X⁷, X⁸, R¹, R², R³, m, n, and p are as defined as in formula I, or salts, or hydrates thereof, processes for their preparation and new intermediates used in the preparation thereof, pharmaceutical compositions containing said compounds and to the use of said compounds in therapy.

### FIELD OF THE INVENTION

The present invention relates to a new class of compounds, to pharmaceutical compositions containing said compounds and to the use of said compounds in therapy. The present invention further relates to processes for the preparation of said compounds and to new intermediates used in the preparation thereof.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999).

Eight distinct mGluR subtypes, termed mGluR1 through mGluR8, have been identified by molecular cloning. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium.

Neurological, Psychiatric and Pain Disorders.

Attempts at elucidating the physiological roles of Group I mGluRs suggest that activation of these receptors elicits neuronal excitation. Various studies have demonstrated that Group I mGluRs agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1(1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated. Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors have been suggested to play roles in a variety of acute and chronic pathophysiological processes and disorders affecting the CNS. These include stroke, head trauma, anoxic and ischemic injuries, hypoglycemia, epilepsy, neurodegenerative disorders such as Alzheimer's disease, psychiatric disorders and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial in all conditions underlain by excessive glutamate-induced excitation of CNS neurons, specifically as neuroprotective agents, analgesics or anticonvulsants.

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

Gastro Intestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “G.I. reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind G.I. reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for treatment of gastro-esophageal reflux disorder (GERD).

The wording “TLESR”, transient lower esophageal sphincter relaxations, is herein defined in accordance with Mittal, R. K, Holloway, R. H., Penagini, R., Blackshaw, L. A., Dent, J., 1995; Transient lower esophageal sphincter relaxation, Gastroenterology 109, pp. 601-610.

The wording “G.I. reflux” is herein defined as fluid from the stomach being able to pass into the esophagus, since the mechanical barrier is temporarily lost at such times.

The wording “GERD”, gastro-esophageal reflux disease, is herein defined in accordance with van Heerwarden, M. A., Smout A. J. P. M, 2000; Diagnosis of reflux disease. Baillière 's Clin. Gastroenterol. 14, pp. 759-774.

Because of their physiological and pathophysiological significance, there is a need for new potent mGluR agonists and antagonists that display a high selectivity for mGluR subtypes, particularly the Group I receptor subtype.

### SUMMARY OF THE INVENTION

In one aspect of the invention there is provided a compound according to formula I 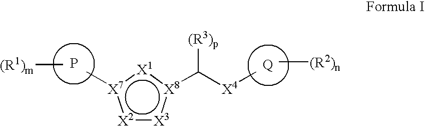
wherein,

P is selected from aryl and heteroaryl

R¹ is attached to P via a carbon atom on ring P and is selected from the group consisting of hydrogen, hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶,C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing atoms independently selected from the group consisting of C, N, O and S;

R⁵ and R⁶ are independently selected from a group consisting of hydrogen, C₁₋₆alkyl, C₃₋₇cycloalkyl and aryl;

X¹, X², and X³, are independently selected from the group consisting of CR⁴, N, O and S;

wherein at least one of X¹, X², and X³ is not N;

X⁷ and X⁸ are selected from the group consisting of C and N such that when X⁷ is N, X⁸ is C and when X⁷ is C, X⁸ is N;

R⁴ is selected from the group consisting of H, ═O, C₁₋₆alkyl, OH;

X⁴ is selected from the group consisting of CR⁷R⁸, NR⁷, O, S, SO, and SO₂;

R⁷ and R⁸ are independently selected from a group consisting of hydrogen, C₁₋₆alkyl, C₃₋₇cycloalkyl and aryl;

R³ is selected from the group consisting of H, C₁₋₆alkyl, hydroxy, C₀₋₆alkylcyano, oxo, ═NR⁵, ═NOR⁵, C₁₋₄alkylhalo, halo, C3-7cycloalkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, OC₁₋₄alkyl, C₁₋₄alkylOR⁵ and C₀₋₄alkylNR⁵R⁶;

R³ can optionally bond to the ring Q to form a fused cyclic group;

R⁷ or R⁸ can optionally bond to R³ or to the ring Q to form a cyclic or a fused cyclic group respectively;

ring Q has 5- to 7-members and may be cycloalkyl, heterocycloalkyl, aryl, or heteroaryl;

R² is selected from the group consisting of hydroxy, C₀₋₆alkylcyano, ═NR⁵, ═NOR⁵, C₁₋₄alkylhalo, halo, C₁₋₆alkyl, C₃₋₆cycloalkyl, C₀₋₆alkylaryl, C₀₋, ₆alkylheteroaryl, C₀₋₆alkylcycloalkyl, C₀₋₆alkylheterocycloalkyl, OC₁₋₄alkyl, OC₀₋₆alkylaryl, O(CO)C₁₋₄alkyl, (CO)OC₁₋₄alkyl, C₀₋₄alkyl(S)C₀₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₁₋₄alkylOR⁵, C₀₋₄alkylNR⁵R⁶ and a 5- or 6-membered ring containing atoms independently selected from C, N, O and S, which ring may optionally be fused with a 5- or 6-membered ring containing one or more atoms independently selected from the group consisting of C, N and O and wherein said ring and said fused ring may be substituted by one or more A;

wherein any C₁₋₆alkyl, aryl or heteroaryl defined under R¹, R² and R³ may be substituted by one or more A;

A is selected from the group consisting of hydrogen, hydroxy, halo, nitro, oxo, C₀₋₆alkylcyano, C₀₋₄alkylC₃₋₆cycloalkyl, C₁₋₆alkyl, C₁₋₆alkylhalo, OC₁₋₆alkylhalo, C₂₋₆alkenyl, C₀₋₃alkylaryl, C₀₋₆alkylOR⁵, OC₂₋₆alkylOR⁵, C₁₋₆alkylSR⁵, OC₂₋₆alkylSR⁵, (CO)R⁵, O(CO)R⁵, OC₂₋₆alkylcyano, OC₁₋₆alkylCO₂R⁵, O(CO)OR⁵, OC₁₋₆alkyl(CO)R⁵, C₁₋₆alkyl(CO)R⁵, NR⁵OR⁶, C₁₋₆alkylNR⁵R⁶, OC₂₋₆alkylNR⁵R⁶, C₀₋₆alkyl(CO)NR⁵R⁶, OC₁₋₆alkyl(CO)NR⁵R⁶, OC₂₋₆alkylNR⁵(CO)R⁶, C₀₋₆alkylNR⁵(CO)R⁶, C₀₋₆alkylNR⁵(CO)NR⁵R⁶, O(CO)NR⁵R⁶, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, SO₃R⁵, C₁₋₆alkylNR⁵(SO₂)NR⁵R⁶, OC₂₋₆alkyl(SO₂)R⁵, C₀₋₆alkyl(SO₂)R⁵, C₀₋₆alkyl(SO)R⁵, OC₂₋₆alkyl(SO)R⁵ and a 5- or 6-membered ring containing atoms independently selected from the group consisting of C, N, O and S;

m is selected from 0, 1, 2, 3 and 4;

n is selected from 0, 1, 2, 3 and 4; and

a salt or hydrate thereof.

In another aspect of the invention there is provided a compound of Formula II 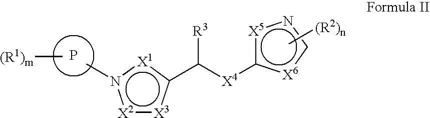
wherein,

P is selected from aryl and heteroaryl;

R¹ is attached to P via a carbon atom on ring P and is selected from the group consisting of hydrogen, hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶,C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing atoms independently selected from the group consisting of C, N, O and S;

R⁵ and R⁶ are independently selected from a group consisting of hydrogen, C₁₋₆alkyl, C₃₋₇cycloalkyl and aryl;

X¹ and X² are independently selected from the group consisting of CR⁴, and N;

X³ is selected from the group consisting of CR⁴, N, and O; wherein at least one of X¹ X² and X³ is not N;

R⁴ is selected from the group consisting of H, ═O, C₁₋₆alkyl, OH;

R³ is selected from the group consisting of H, C₁₋₆alkyl, hydroxy, C₀₋₆alkylcyano, oxo, ═NR⁵, ═NOR⁵, C₁₋₄alkylhalo, halo, C3-7cycloalkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, OC₁₋₄alkyl, C₁₋₄alkylOR⁵ and C₀₋₄alkylNR⁵R⁶;

X⁴ is selected from the group consisting of CR⁷R⁸, NR⁷, O, S, SO, and SO₂;

R⁷ and R⁸ are independently selected from a group consisting of hydrogen, C₁₋₆alkyl, C₃₋₇cycloalkyl and aryl;

X⁵ and X⁶ are independently selected from the group consisting of C, N, O and S;

R² is selected from the group consisting of hydroxy, C₀₋₆alkylcyano, ═NR⁵, NOR⁵, C₁₋₄alkylhalo, halo, C₁₋₆alkyl, C₃₋₆cycloalkyl, C₀₋₆alkylaryl, C₀₋, ₆alkylheteroaryl, C₀₋₆alkylcycloalkyl, C₀₋₆alkylheterocycloalkyl, OC₁₋₄alkyl, OC₀₋₆alkylaryl, O(CO)C₁₋₄alkyl, (CO)OC₁₋₄alkyl, C₀₋₄alkyl(S)C₀₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₁₋₄alkylOR⁵, C₀₋₄alkylNR⁵R⁶ and a 5- or 6-membered ring containing atoms independently selected from C, N, O and S, and wherein said ring may be substituted by one or more A; and any C₁₋₆alkyl, aryl or heteroaryl defined under R¹, R² and R³ may be substituted by one or more A;

A is selected from the group consisting of hydrogen, hydroxy, halo, nitro, oxo, C₀₋₆alkylcyano, C₀₋₄alkylC₃₋₆cycloalkyl, C₁₋₆alkyl, C₁₋₆alkylhalo, OC₁₋₆alkylhalo, C₂₋₆alkenyl, C₀₋₃alkylaryl, C₀₋₆alkylOR⁵, OC₂₋₆alkylOR⁵, C₁₋₆alkylSR⁵, OC₂₋₆alkylSR⁵, (CO)R⁵, O(CO)R⁵, OC₂₋₆alkylcyano, OC₁₋₆alkylCO₂R⁵, O(CO)OR⁵, OC₁₋₆alkyl(CO)R⁵, C₁₋₆alkyl(CO)R⁵, NR⁵OR⁶, C₁₋₆alkylNR⁵R⁶, OC₂₋₆alkylNR⁵R⁶, C₀₋₆alkyl(CO)NR⁵R⁶, OC₁₋₆alkyl(CO)NR⁵R⁶, OC₂₋₆alkylNR⁵(CO)R⁶, C₀₋₆alkylNR⁵(CO)R⁶, C₀₋₆alkylNR⁵(CO)NR⁵R⁶, O(CO)NR⁵R⁶, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, SO₃R⁵, C₁₋₆alkylNR⁵(SO₂)NR⁵R⁶, OC₂₋₆alkyl(SO₂)R⁵, C₀₋₆alkyl(SO₂)R⁵, C₀₋₆alkyl(SO)R⁵, OC₂₋₆alkyl(SO)R⁵ and a 5- or 6-membered ring containing atoms independently selected from the group consisting of C, N, O and S;

m is selected from 0, 1, 2, 3 and 4;

n is selected from 0, 1, 2, 3 and 4;

p is selected from 1 and 2; and

a salts or hydrates thereof,

In a further aspect of the invention there is provided pharmaceutical compositions comprising a therapeutically effective amount of a compound of formula I or formula II and a pharmaceutically acceptable diluent, excipients and/or inert carrier.

In yet a further aspect of the invention there is provided a pharmaceutical composition comprising a compound of formula I, or formula II for use in the treatment of mGluR5 receptor mediated disorders, and for use in the treatment of neurological disorders, psychiatric disorders, gastrointestinal disorders and pain disorders.

In still a further aspect of the invention there is provided the compound of formula I or formula II for use in therapy, especially for the treatment of mGluR5 receptor mediated disorders, and for the treatment of neurological disorders, psychiatric disorders, gastrointestinal disorders and pain disorders.

In another aspect of the invention there is provided processes for the preparation of compounds of formula I and formula II and the intermediates used in the preparation thereof.

A further aspect of the invention is the use of a compound according to formula I for the manufacture of a medicament for the treatment or prevention of obesity and obesity related conditions, as well as treating eating disorders by inhibition of excessive food intake and the resulting obesity and complications associated therewith.

These and other aspects of the present invention are described in greater detail herein below.

### DETAILED DESCRIPTION OF THE INVENTION

The object of the present invention is to provide compounds exhibiting an activity at metabotropic glutamate receptors (mGluRs), especially at the mGluR5 receptors.

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms. Similarly ‘C₁₋₃’ means a carbon group having 1, 2, or 3 carbon atoms

In the case where a subscript is the integer 0 (zero) the group to which the subscript refers indicates that the group is absent.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl. The term C₁₋₃alkyl has 1 to 3 carbon atoms and may be methyl, ethyl, n-propyl or i-propyl.

In this specification, unless stated otherwise, the term “cycloalkyl” refers to an optionally substituted, saturated cyclic hydrocarbon ring system. The term “C₃₋₇cycloalkyl” may be cyclopropyl, cyclobutyl, cyclopentyl, cyclohexyl or cycloheptyl.

In this specification, unless stated otherwise, the term “alkoxy” includes both straight or branched alkoxy groups. C₁₋₃alkoxy may be, but is not limited to methoxy, ethoxy, n-propoxy or i-propoxy.

In this specification, unless stated otherwise, the term “bond” may be a saturated or unsaturated bond.

In this specification, unless stated otherwise, the term “halo” and “halogen” may be fluoro, chloro, bromo or iodo.

In this specification, unless stated otherwise, the term “alkylhalo” means an alkyl group as defined above, which is substituted with halo as described above. The term “C₁₋₆alkylhalo” may include, but is not limited to fluoromethyl, difluoromethyl, trifluoromethyl, fluoroethyl, difluoroethyl or bromopropyl. The term “OC₁₋₆alkylhalo” may include, but is not limited to fluoromethoxy, difluoromethoxy, trifluoromethoxy, fluoroethoxy or difluoroethoxy.

In this specification, unless stated otherwise, the term “alkenyl” includes both straight and branched chain alkenyl groups. The term “C₂₋₆alkenyl” refers to an alkenyl group having 2 to 6 carbon atoms and one or two double bonds, and may be, but is not limited to vinyl, allyl, propenyl, i-propenyl, butenyl, i-butenyl, crotyl, pentenyl, i-pentenyl and hexenyl.

In this specification, unless stated otherwise, the term “alkynyl” includes both straight and branched chain alkynyl groups. The term C₂₋₆alkynyl having 2 to 6 carbon atoms and one or two triple bonds, and may be, but is not limited to ethynyl, propargyl, butynyl, i-butynyl, pentynyl, i-pentynyl and hexynyl.

In this specification unless otherwise stated the term “aryl” refers to an optionally substituted monocyclic or bicyclic hydrocarbon ring system containing at least one unsaturated aromatic ring. Examples and suitable values of the term “aryl” are phenyl, naphthyl, 1,2,3,4-tetrahydronaphthyl, indyl and indenyl.

In this specification, unless stated otherwise, the term “heteroaryl” refers to an optionally substituted monocyclic or bicyclic unsaturated, ring system containing at least one heteroatom selected independently from N, O or S. Examples of “heteroaryl” may be, but are not limited to thiophene, thienyl, pyridyl, thiazolyl, furyl, pyrrolyl, triazolyl, imidazolyl, oxadiazolyl, oxazolyl, isoxazolyl, pyrazolyl, imidazolonyl, oxazolonyl, thiazolonyl, tetrazolyl and thiadiazolyl, benzoimidazolyl, benzooxazolyl, tetrahydrotriazolopyridyl, tetrahydrotriazolopyrimidinyl, benzofuryl, indolyl, isoindolyl, pyridonyl, pyridazinyl, pyrimidinyl, imidazopyridyl, oxazolopyridyl, thiazolopyridyl, pyridyl, imidazopyridazinyl, oxazolopyridazinyl, thiazolopyridazinyl and purinyl.

In this specification, unless stated otherwise, the term “alkylaryl”, “alkylheteroaryl” and “alkylcycloalkyl” refer to a substituent that is attached via the alkyl group to an aryl, heteroaryl and cycloalkyl group.

In this specification, unless stated otherwise, the term “heterocycloalkyl” refers to an optionally substituted, saturated cyclic hydrocarbon ring system wherein one or more of the carbon atoms are replaced with heteroatom. The term “heterocycloalkyl” includes but is not limited to pyrrolidine, tetrahydrofuran, tetrahydrothiophene, piperidine, piperazine, morpholine, thiomorpholine, tetrahydropyran, tetrahydrothiopyran.

In this specification, unless stated otherwise the term “5- or 6-membered ring containing atoms independently selected from C, N, O or S”, includes aromatic and heteroaromatic rings as well as carbocyclic and heterocyclic rings, which may be saturated partially saturated or unsaturated. Examples of such rings may be, but are not limited to furyl, isoxazolyl, isothiazolyl, oxazolyl, pyrazinyl, pyrazolyl, pyridazinyl, pyridyl, pyrimidyl, pyrrolyl, thiazolyl, thienyl, imidazolyl, imidazolidinyl, imidazolinyl, triazolyl, morpholinyl, piperazinyl, piperidyl, piperidonyl, pyrazolidinyl, pyrazolinyl, pyrrolidinyl, pyrrolinyl, tetrahydropyranyl, thiomorpholinyl, phenyl, cyclohexyl, cyclopentyl and cyclohexenyl.

In this specification, unless stated otherwise, the term “═NR⁵” and “═NOR⁵” include imino- and oximo- groups carrying an R⁵ substituent and may be, or be part of, groups including, but not limited to iminoalkyl, iminohydroxy, iminoalkoxy, amidine, hydroxyamidine and alkoxyamidine.

In the case where a subscript is the integer 0 (zero) the group to which the subscript refers, indicates that the group is absent, i.e. there is a direct bond between the groups. In this specification unless stated otherwise the term “fused rings” refers to two rings which share 2 common atoms.

In this specification, unless stated otherwise, the term “bridge” means a molecular fragment, containing one or more atoms, or a bond, which connects two remote atoms in a ring, thus forming either bi- or tricyclic systems.

One embodiment of the invention relates to compounds of Formula I 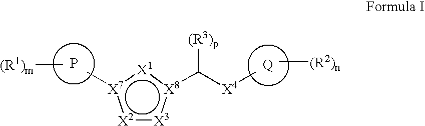
wherein,

P is selected from aryl and heteroaryl

R¹ is attached to P via a carbon atom on ring P and is selected from the group consisting of hydrogen, hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing one or more atoms independently selected from the group consisting of C, N, O and S;

R⁵ and R⁶ are independently selected from a group consisting of hydrogen, C₁₋₆alkyl, C₃₋₇cycloalkyl and aryl;

X¹, X², and X³, are independently selected from the group consisting of CR⁴, N, O and S;

wherein at least one of X¹, X², and X³ is not N;

X⁷ and X⁸ are selected from the group consisting of C and N such that when X⁷ is N, X⁸ is C and when X⁷ is C, X⁸ is N;

R⁴ is selected from the group consisting of H, ═O, C₁₋₆alkyl, OH;

X⁴ is selected from the group consisting of CR⁷R⁸, NR⁷, O, S, SO, and SO₂;

R⁷ and R⁸ are independently selected from a group consisting of hydrogen, C₁₋₆alkyl, C₃₋₇cycloalkyl and aryl;

R³ is selected from the group consisting of H, C₁₋₆alkyl, hydroxy, C₀₋₆alkylcyano, oxo, ═NR⁵, ═NOR⁵, C₁₋₄alkylhalo, halo, C3-7cycloalkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, OC₁₋₄alkyl, C₁₋₄alkylOR⁵ and C₀₋₄alkylNR⁵R⁶;

R³ can optionally bond to the ring Q to form a fused cyclic group;

R⁷ or R⁸ can optionally bond to R³ or to the ring Q to form a cyclic or a fused cyclic group respectively;

ring Q has 5- to 7-members and may be carbocyclic, heterocyclic, aryl heteroaryl;

R² is selected from the group consisting of hydroxy, C₀₋₆alkylcyano, ═NR⁵, ═NOR⁵, C₁₋₄alkylhalo, halo, C₁₋₆alkyl, C₃₋₆cycloalkyl, C₀₋₆alkylaryl, C₀₋, ₆alkylheteroaryl, C₀₋₆alkylcycloalkyl, C₀₋₆alkylheterocycloalkyl, OC₁₋₄alkyl, OC₀₋₆alkylaryl, O(CO)C₁₋₄alkyl, (CO)OC₁₋₄alkyl, C₀₋₄alkyl(S)C₀₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₁₋₄alkylOR⁵, C₀₋₄alkylNR⁵R⁶ and a 5- or 6-membered ring containing one or more atoms independently selected from C, N, O and S, which ring may optionally be fused with a 5- or 6-membered ring containing one or more atoms independently selected from the group consisting of C, N and O and wherein said ring and said fused ring may be substituted by one or more A;

wherein any C₁₋₆alkyl, aryl or heteroaryl defined under R¹, R² and R³ may be substituted by one or more A;

A is selected from the group consisting of hydrogen, hydroxy, halo, nitro, oxo, C₀₋₆alkylcyano, C₀₋₄alkylC₃₋₆cycloalkyl, C₁₋₆alkyl, C₁₋₆alkylhalo, OC₁₋₆alkylhalo, C₂₋₆alkenyl, C₀₋₃alkylaryl, C₀₋₆alkylOR⁵, OC₂₋₆alkylOR⁵, C₁₋₆alkylSR⁵, OC₂₋₆alkylSR⁵, (CO)R⁵, O(CO)R⁵, OC₂₋₆alkylcyano, OC₁₋₆alkylCO₂R⁵, O(CO)OR⁵, OC₁₋₆alkyl(CO)R⁵, C₁₋₆alkyl(CO)R⁵, NR⁵OR⁶, C₁₋₆alkylNR⁵R⁶, OC₂₋₆alkylNR⁵R⁶, C₀₋₆alkyl(CO)NR⁵R⁶, OC₁₋₆alkyl(CO)NR⁵R⁶, OC₂₋₆alkylNR⁵(CO)R⁶, C₀₋₆alkylNR⁵(CO)R⁶, C₀₋₆alkylNR⁵(CO)NR⁵R⁶, O(CO)NR⁵R⁶, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, SO₃R⁵, C₁₋₆alkylNR⁵(SO₂)NR⁵R⁶, OC₂₋₆alkyl(SO₂)R⁵, C₀₋₆alkyl(SO₂)R⁵, C₀₋₆alkyl(SO)R⁵, OC₂₋₆alkyl(SO)R⁵ and a 5- or 6-membered ring containing one or more atoms independently selected from the group consisting of C, N, O and S;

m is selected from 0, 1, 2, 3 and 4;

n is selected from 0, 1, 2, 3 and 4;

p is selected from 1 and 2; and

a salt or hydrate thereof.

Another embodiment of the invention relates to compounds of Formula II 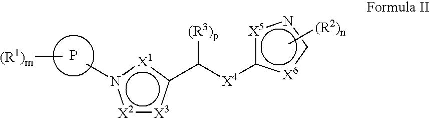
wherein,

P is selected from aryl and heteroaryl;

R¹ is attached to P via a carbon atom on ring P and is selected from the group consisting of hydrogen, hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶,C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing one or more atoms independently selected from the group consisting of C, N, O and S;

R⁵ and R⁶ are independently selected from a group consisting of hydrogen, C₁₋₆alkyl, C₃₋₇cycloalkyl and aryl;

X¹ and X² are independently selected from the group consisting of CR⁴, and N;

X³ is selected from the group consisting of CR⁴, N, and O; wherein at least one of X¹ X² and X³ is not N;

R⁴ is selected from the group consisting of H, ═O, C₁₋₆alkyl, OH;

R³ is selected from the group consisting of H, C₁₋₆alkyl, hydroxy, C₀₋₆alkylcyano, oxo, ═NR⁵, ═NOR⁵, C₁₋₄alkylhalo, halo, C3-7cycloalkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, OC₁₋₄alkyl, C₁₋₄alkylOR⁵ and C₀₋₄alkylNR⁵R⁶;

X⁴ is selected from the group consisting of CR⁷R⁸, NR⁷, O, S, SO, and SO₂;

R⁷ and R⁸ are independently selected from a group consisting of hydrogen, C₁₋₆alkyl, C₃₋₇cycloalkyl and aryl;

X⁵ and X⁶ are independently selected from the group consisting of C, N, O and S;

R² is selected from the group consisting of hydrogen, hydroxy, C₀₋₆alkylcyano, ═NR⁵, ═NOR⁵, C₁₋₄alkylhalo, halo, C₁₋₆alkyl, C₃₋₆cycloalkyl, C₀₋₆alkylaryl, C₀, ₆alkylheteroaryl, C₀₋₆alkylcycloalkyl, C₀₋₆alkylheterocycloalkyl, OC₁₋₄alkyl, OC₀₋₆alkylaryl, O(CO)C₁₋₄alkyl, (CO)OC₁₋₄alkyl, C₀₋₄alkyl(S)C₀₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₁₋₄alkylOR⁵, C₀₋₄alkylNR⁵R⁶ and a 5- or 6-membered ring containing one or more atoms independently selected from C, N, O and S, and wherein said ring may be substituted by one or more A; and

any C₁₋₆alkyl, aryl or heteroaryl defined under R¹, R² and R³ may be substituted by one or more A;

A is selected from the group consisting of hydrogen, hydroxy, halo, nitro, oxo, C₀₋₆alkylcyano, C₀₋₄alkylC₃₋₆cycloalkyl, C₁₋₆alkyl, C₁₋₆alkylhalo, OC₁₋₆alkylhalo, C₂₋₆alkenyl, C₀₋₃alkylaryl, C₀₋₆alkylOR⁵, OC₂₋₆alkylOR⁵, C₁₋₆alkylSR⁵, OC₂₋₆alkylSR⁵, (CO)R⁵, O(CO)R⁵, OC₂₋₆alkylcyano, OC₁₋₆alkylCO₂R⁵, O(CO)OR⁵, OC₁₋₆alkyl(CO)R⁵, C₁₋₆alkyl(CO)R⁵, NR⁵OR⁶, C₁₋₆alkylNR⁵R⁶, OC₂₋₆alkylNR⁵R⁶, C₀₋₆alkyl(CO)NR⁵R⁶, OC₁₋₆alkyl(CO)NR⁵R⁶, OC₂₋₆alkylNR⁵(CO)R⁶, C₀₋₆alkylNR⁵(CO)R⁶, C₀₋₆alkylNR⁵(CO)NR⁵R⁶, O(CO)NR⁵R⁶, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, SO₃R⁵, C₁₋₆alkylNR⁵(SO₂)NR⁵R⁶, OC₂₋₆alkyl(SO₂)R⁵, C₀₋₆alkyl(SO₂)R⁵, C₀₋₆alkyl(SO)R⁵, OC₂₋₆alkyl(SO)R⁵ and a 5- or 6-membered ring containing one or more atoms independently selected from the group consisting of C, N, O and S;

m is selected from 0, 1, 2, 3 and 4;

n is selected from 0, 1, 2, 3 and 4;

p is selected from 1 and 2; and

and a salts or hydrates thereof,

Another embodiment the invention relates to the compounds:

3-(3-chlorophenyl)-5-{[(4-methyl-5-pyridin-3-yl-4H-1,2,4-triazol-3-yl)thio]methyl}-1,3,4-oxadiazol-2(3H)-one

2-(3-chlorophenyl)-5-{1-[methyl(4-methyl-5-pyridin-4-yl-4H-1,2,4-triazol-3-yl)amino]ethyl}-2,4-dihydro-3H-1,2,4-triazol-3-one

4-(5-{1-[1-(3-chlorophenyl)-1H-pyrazol-4-yl]ethoxy}-4-methyl-4H-1,2,4-triazol-3-yl)pyridine

4-(5-{1-[2-(3-chlorophenyl)-2H-1,2,3-triazol-4-yl]ethoxy}-4-methyl-4H-1,2,4-triazol-3-yl)pyridine

4-[5-({1-[2-(3-chlorophenyl)-2H-1,2,3-triazol-4-yl]ethyl}thio)-4-cyclopropyl-4H-1,2,4-triazol-3-yl]pyridine

4-{5-[1-(3-Chloro-phenyl)-1H-[1,2,4]triazol-3-ylmethylsulfanyl]-4-cyclopropyl-4H-[1,2,4]triazol-3-yl}-pyridine

4-{5-[1-(3-Chloro-phenyl)-1H-[1,2,4]triazol-3-ylmethoxy]-4-cyclopropyl-4H-[1,2,4]triazol-3-yl}-pyridine

4-{5-[1-(3-Chloro-phenyl)-1H-[1,2,3]triazol-4-ylmethylsulfanyl]-4-methyl-4H-[1,2,4]triazol-3-yl}-pyridine

4-{5-[1-(3-Chloro-phenyl)-1H-[1,2,3]triazol-4-ylmethylsulfanyl]-4-cyclopropyl-4H-[1,2,4]triazol-3-yl}-pyridine

4-{5-[1-(3-Chloro-phenyl)-1H-[1,2,3]triazol-4-ylmethoxy]-4-cyclopropyl-4H-[1,2,4]triazol-3-yl}-pyridine and

4-(5-{(1R)-[2-(3-chlorophenyl)-2H-1,2,3-triazol-4-yl]ethoxy}-4-methyl-4H-1,2,4-triazol-3-yl)pyridine

or a salt or hydrate thereof.

This invention relates to triazoles and other heterocyclic compounds of formulas I and II, having a variable P. In one embodiment of the invention P is selected from aryl and heteroaryl. In another embodiment P is aryl and in still another embodiment P is phenyl.

According to Formulas I and II, P can be substituted with 0 to 4 substituents R¹. In one embodiment of the invention P has at least one substituent R¹. In one embodiment of the invention P has one substituent R¹. In a preferred embodiment, the substituent R¹ is at the meta position relative to X⁷. In another embodiment of the invention R1 has 2 substituents R¹. In a preferred embodiment the substituents R¹ are in the 2-position (meta) and 5-position (ortho) to X⁷. In one embodiment of the invention R1 is selected from hydrogen, hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶,C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, (CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing one or more atoms independently selected from the group consisting of C, N, O and S. In another embodiment of the invention R¹ is selected from halo, C₁₋₆alkyl, —OC₁₋₆alkyl, C₀₋₆alkylcyano. In another embodiment R¹ is selected from Cl, F, CN and methyl.

Embodiments of the invention include those wherein R⁵ and R⁶ are selected from hydrogen, C₁₋₆alkyl, C₃₋₇cycloalkyl and aryl.

Formula I allows for variables X⁷ and X⁸. In one embodiment of the invention X⁷ and X⁸ are selected from C and N, such that when X⁷ is N, X⁸ is C and when X⁷ is C, X⁸ is N.

Formulas I and II provide variables X¹, X² and X³. In one embodiment of the invention X¹, X² and X³ are independently selected from CR⁴, N, O and S such that at least one of X¹, X², and X³ is not N. In another embodiment of the invention at least one of X¹, X² and X³ is not CR⁴. In another embodiment of the invention X¹ and X² are independently selected from the group consisting of CR⁴, and N, and X³ is selected from the group consisting of CR⁴, N, and O such that at least one of X¹ X² and X³ is not N.

In still another embodiment of the invention X¹ X² and X³ are selected such that the ring that they form is one of:

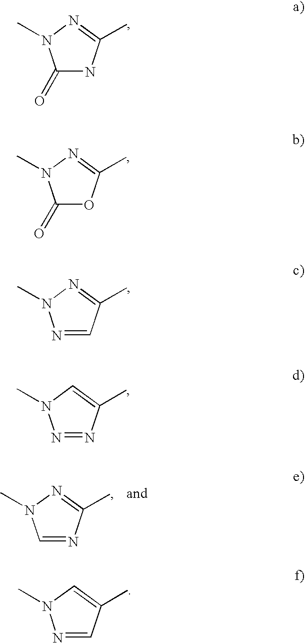

In still a further embodiment of the invention X¹ X² and X³ are selected such that the ring that they form is one of:

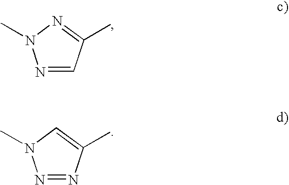

When X¹, X² or X³ is CR⁴, the variable R⁴ is selected from H, ═O, C₁₋₆alkyl, OH. In particular embodiments R⁴ is H, ═O. In a preferred embodiment R⁴ is H. A linker group comprised of a carbon atom and a variable X⁴, joins the five membered ring containing variables X¹ X² and X³ to the ring Q. The carbon atom has one or two substituents R³ which are independently selected from H, C₁₋₆alkyl, hydroxy, C₀₋₆alkylcyano, oxo, ═NR⁵, ═NOR⁵, C₁₋₄alkylhalo, halo, C3-7cycloalkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, OC₁₋₄alkyl, C₁₋₄alkylOR⁵ and C₀₋₄alkylNR⁵R⁶. In a preferred embodiment R³ is selected from the group consisting of H and C₁₋₆alkyl. Preferably R³ is H or methyl.

The variable X⁴ is selected from CR⁷R⁸, NR⁷, O, S, SO, and SO₂. In a particular embodiment X⁴ is selected from CR⁷R⁸, NR⁷, O, S. The variables R⁷ and R⁸ are independently selected from hydrogen, C₁₋₆alkyl, C₃₋₇cycloalkyl and aryl. In one embodiment R⁷ and R⁸ are independently selected from hydrogen and C₁₋₆alkyl. In particular embodiments R⁷ and R⁸ are independently selected from hydrogen and methyl.

In embodiments of the invention, R³ can optionally bond to the ring Q, thereby forming a fused cyclic group.

In other embodiments of the invention R⁷ or R⁸ can optionally bond to R³ to form a cyclic group.

In still other embodiments of the invention R⁷ or R⁸ can optionally bond to Q to form a fused cyclic group.

Formula 1 provides a ring Q, which contains 5- to 7-members and may be cycloalkyl, heterocycloalkyl, aryl or heteroaryl. In particular embodiments of the invention the ring Q is a 5-memebred ring. In more particular embodiments of the invention Q is a heteroaromatic ring. In still more particular embodiments of the invention Q is: 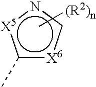
as shown in formula II.

As provided in formula II the ring contains two variables X⁵ and X⁶. In embodiments of the invention X⁵ and X⁶ are independently selected from C, N, O and S. In one preferred embodiment of the invention X⁵ and X⁶ are both N. In another embodiment X⁵ is C and X⁶ is N. In still another preferred embodiment X⁵ is N and X⁶ is O.

Formulas I and II allow for 0 to 4 variables R² on the ring Q or the ring containing X⁵ and X⁶, respectively. In one embodiment of the invention there is provided one variable R². In another embodiment of the invention there is provided two variables R². The variables, R² are independently selected from hydrogen, hydroxy, C₀₋₆alkylcyano, ═NR⁵, NOR⁵, C₁₋₄alkylhalo, halo, C₁₋₆alkyl, C₃₋₆cycloalkyl, C₀₋₆alkylaryl, C₀₋₆alkylheteroaryl, C₀₋₆alkylcycloalkyl, C₀₋₆alkylheterocycloalkyl, OC₁₋₄alkyl, OC₀₋₆alkylaryl, O(CO)C₁₋₄alkyl, (CO)OC₁₋₄alkyl, C₀₋₄alkyl(S)C₀₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₁₋₄alkylOR⁵, C₀₋₄alkylNR⁵R⁶ and a 5- or 6-membered ring containing one or more atoms independently selected from C, N, O and S, which ring may optionally be fused with a 5- or 6-membered ring containing atoms independently selected from the group consisting of C, N and O and wherein said ring and said fused ring may be substituted by one or more A; In a preferred embodiment of the invention the variable R² is selected from H, C₁₋₆alkyl, C₃₋₆cycloalkyl, C₀₋₆alkylaryl, C₃₋₆cycloalkyl and C_{0-,6}alkylheteroary. In a preferred embodiment of the invention there is a variable R² that is selected from C₀₋₆alkylaryl, and C_{0,-6}alkylheteroary, more preferably from aryl and heteroaryl and still more preferably from 4-pyridyl, 3-pyridyl and phenyl. In another preferred embodiment when there are two variables R² the first is selected from the group aryl and heteroaryl, and the second is selected from C¹⁻⁶alkyl and C₃₋₆cycloalkyl. In another preferred embodiment of the invention one variable is 4-pyridyl and the other is methyl. In another preferred embodiment of the invention one variable is 4-pyridyl and the other is cyclopropyl.

Formulas I and II further allow the variable R² and any C₁₋₆alkyl, aryl, or heteroaryl group defined under R¹ and R³ to be further substituted with one or more variables A. The variables A are independently selected from hydrogen, hydroxy, halo, nitro, oxo, C₀₋₆alkylcyano, C₀₋₄alkylC₃₋₆cycloalkyl, C₁₋₆alkyl, C₁₋₆alkylhalo, OC₁₋₆alkylhalo, C₂₋₆alkenyl, C₀₋₃alkylaryl, C₀₋₆alkylOR⁵, OC₂₋₆alkylOR⁵, C₁₋₆alkylSR⁵, OC₂₋₆alkylSR⁵, (CO)R⁵, O(CO)R⁵, OC₂₋₆alkylcyano, OC₁₋₆alkylCO₂R⁵, O(CO)OR⁵, OC₁₋₆alkyl(CO)R⁵, C₁₋₆alkyl(CO)R⁵, NR⁵OR⁶, C₁₋₆alkylNR5R6, OC₂₋₆alkylNR⁵R⁶, C₀₋₆alkyl(CO)NR⁵R⁶, OC₁₋₆alkyl(CO)NR⁵R⁶, OC₂₋₆alkylNR⁵(CO)R⁶, C₀₋₆alkylNR⁵(CO)R⁶, C₀₋₆alkylNR⁵(CO)NR⁵R⁶, O(CO)NR⁵R⁶, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, SO₃R⁵, C₁₋₆alkylNR⁵(SO₂)NR⁵R⁶, OC₂₋₆alkyl(SO₂)R⁵, C₀₋₆alkyl(SO₂)R⁵, C₀₋₆alkyl(SO)R⁵, OC₂₋₆alkyl(SO)R⁵ and a 5- or 6-membered ring containing atoms independently selected from the group consisting of C, N, O and S. In further embodiment of the invention A is selected from Cl, F, CN, Me, OMe, and OH.

Embodiments of the invention include salt forms of the compounds of Formula I and II. Salts for use in pharmaceutical compositions will be pharmaceutically acceptable salts, but other salts may be useful in the production of the compounds of Formula I.

A suitable pharmaceutically acceptable salt of the compounds of the invention is, for example, an acid-addition salt, for example an inorganic or organic acid. In addition, a suitable pharmaceutically acceptable salt of the compounds of the invention is an alkali metal salt, an alkaline earth metal salt or a salt with an organic base.

Other pharmaceutically acceptable salts and methods of preparing these salts may be found in, for example, Remington's Pharmaceutical Sciences (18^{th} Edition, Mack Publishing Co.) 1990.

Some compounds of formula I may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of Formula I and II.

The invention further relates to hydrate and solvate forms of the compounds of Formula I and II

Pharmaceutical Composition

According to one aspect of the present invention there is provided a pharmaceutical composition comprising as active ingredient a therapeutically effective amount of the compound of Formula I or more particularly a compound of Formula II, or salts, solvates or solvated salts thereof, in association with one or more pharmaceutically acceptable diluent, excipients and/or inert carrier.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream or for rectal administration e.g. as a suppository.

In general the above compositions may be prepared in a conventional manner using one or more conventional excipients, pharmaceutical acceptable diluents and/or inert carriers.

Suitable daily doses of the compounds of formula I in the treatment of a mammal, including man are approximately 0.01 to 250 mg/kg bodyweight at peroral administration and about 0.001 to 250 mg/kg bodyweight at parenteral administration.

The typical daily dose of the active ingredients varies within a wide range and will depend on various factors such as the relevant indication, severity of the illness being treated, the route of administration, the age, weight and sex of the patient and the particular compound being used, and may be determined by a physician.

Medical Use

It has been found that the compounds according to the present invention, exhibit a high degree of potency and selectivity for individual metabotropic glutamate receptor (mGluR) subtypes. Accordingly, the compounds of the present invention are expected to be useful in the treatment of conditions associated with excitatory activation of mGluR5 and for inhibiting neuronal damage caused by excitatory activation of mGluR5. The compounds may be used to produce an inhibitory effect of mGluR5 in mammals, including man.

The mGluR Group I receptor including mGluR5 are highly expressed in the central and peripheral nervous system and in other tissues. Thus, it is expected that the compounds of the invention are well suited for the treatment of mGluR5-mediated disorders such as acute and chronic neurological and psychiatric disorders, gastrointestinal disorders, and chronic and acute pain disorders.

The invention relates to compounds of Formula I and Formula II, as defined hereinbefore, for use in therapy.

The invention relates to compounds of Formula I and Formula II, as defined hereinbefore, for use in treatment of mGluR5-mediated disorders.

The invention relates to compounds of Formula I and Formula II, as defined hereinbefore, for use in treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, ophthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of Formula I and Formula II, as defined hereinbefore, for use in treatment of pain related to migraine, inflammatory pain, neuropathic pain disorders such as diabetic neuropathies, arthritis and rheumatoid diseases, low back pain, post-operative pain and pain associated with various conditions including angina, renal or biliary colic, menstruation, migraine and gout.

The invention relates to compounds of Formula I and Formula II as defined hereinbefore, for use in treatment of stroke, head trauma, anoxic and ischemic injuries, hypoglycemia, cardiovascular diseases and epilepsy.

The present invention relates also to the use of a compound of Formula I and Formula II as defined hereinbefore, in the manufacture of a medicament for the treatment of mGluR Group I receptor-mediated disorders and any disorder listed above.

One embodiment of the invention relates to the use of a compound according to Formula I and Formula II in the treatment of gastrointestinal disorders.

Another embodiment of the invention relates to the use of a compound according to Formula I and Formula II, for the manufacture of a medicament for the inhibition of transient lower esophageal sphincter relaxations, for the treatment of GERD, for the prevention of G.I. reflux, for the treatment regurgitation, treatment of asthma, treatment of laryngitis, treatment of lung disease and for the management of failure to thrive.

A further embodiment of the invention is the use of a compound according to formula I for the manufacture of a medicament for the treatment or prevention of functional gastrointestinal disorders, such as functional dyspepsia (FD). Yet another aspect of the invention is the use of a compound according to formula I for the manufacture of a medicament for the treatment or prevention of irritable bowel syndrome (IBS), such as constipation predominant IBS, diarrhea predominant IBS or alternating bowel movement predominant IBS.

A further aspect of the invention is the use of a compound according to formula I for the manufacture of a medicament for the treatment or prevention of obesity and obesity related conditions, as well as treating eating disorders by inhibition of excessive food intake and the resulting obesity and complications associated therewith.

These and other aspects of the present invention are described in greater detail herein below.

The invention also provides a method of treatment of mGluR5-mediated disorders and any disorder listed above, in a patient suffering from, or at risk of, said condition, which comprises administering to the patient an effective amount of a compound of Formula I and Formula II, as hereinbefore defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand.

The term “disorder”, unless stated otherwise, means any condition and disease associated with metabotropic glutamate receptor activity.

Non-Medical Use

In addition to their use in therapeutic medicine, the compounds of Formula I and Formula II, salts or hydrates thereof, are also useful as pharmacological tools in the development and standardisation of in vitro and in vivo test systems for the evaluation of the effects of inhibitors of mGluR related activity in laboratory animals such as cats, dogs, rabbits, monkeys, rats and mice, as part of the search for new therapeutics agents.

Methods of Preparation

Another aspect of the present invention provides processes for preparing compounds of Formula I and II, or salts or hydrates thereof. Processes for the preparation of the compounds in the present invention are described herein.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C.

The term “reflux” shall mean, unless otherwise stated, in reference to an employed solvent a temperature at or above the boiling point of named solvent.

Abbreviations

atm atmosphere

aq. aqueous

CDI N,N′-Carbonyldiimidazole

DCC N,N-Dicyclohexylcarbodiimide

DCM Dichloromethane

DEA N,N-Diisopropyl ethylamine

DIC N,N′-Diisopropylcarbodiimide

DMAP N,N-Dimethyl-4-aminopyridine

DMF N,N-Dimethylformamide

DMSO Dimethylsulfoxide

EA Ethyl acetate

EDCl N-[3-(dimethylamino)propyl]-N′-ethylcarbodiimide hydrochloride

EtOAc Ethyl acetate

Et₂O Diethylether

h hour(s)

HOBt N-Hydroxybenzotriazole

HBTU O-(Benzotriazol-1-yl)-N,N,N′,N′-tetramethyluronium hexafluorophosphate

MCPBA m-chlorbenzoic acid

MeCN acetonitrile

MeOH Methanol

min minutes

nBuLi 1-butyl lithium

Novozyme 435® Polymer supported Candida Antartica Lipase (Novozymes, Bagsvaerd, Denmark)

o.n. over night

RT, rt, r.t. room temperature

TEA Triethylamine

THF Tetrahydrofuran

BOC tert-butoxycarbonyl

nBu normal butyl

EDC 1-Ethyl-3-(3-dimethylaminopropyl)carbodiimide

PPTS pyridinium p-toluenesulfonate

TBAF tetrabutylammonium fluoride

pTsOH p-toluenesulfonic acid

SPE solid phase extraction (usually containing silica gel for mini-chromatography)

sat. saturated

n-BuLi 1-butyllithium

OMs mesylate or methane sulfonate ester

OTs tosylate, toluene sulfonate or 4-methylbenzene sulfonate ester

HetAr heteroaryl

NaOAc sodium acetate

EtOAc ethyl acetate

EtOH ethanol

EtI iodoethane

Et ethyl

MeI iodomethane

MeMgCl methyl magnesium chloride

Me methyl

NMR nuclear magnetic resonance

HPLC high performance liquid chromatography

LCMS HPLC mass spec

Preparation of Intermediates

The intermediates provided in synthetic paths given below, are useful for further preparation of compounds of formula I or II. Other starting materials are either commercially available or can be prepared via methods described in the literature. The synthetic pathways described below are non-limiting examples of preparations that can be used. One of skill in the art would understand other pathways might be used.

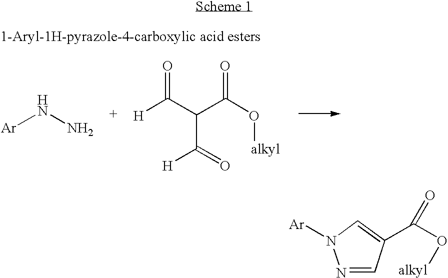

With reference to scheme 1, pyrazoles carboxylic acid esters may be obtained by reaction of 3-arylhydrazines with alkyl 2-formyl-3-oxopropanoate in solvents such ethanol at temperatures from 40 to 140° C. [Holzer, W.; Seiringer, G.; J.Heterocycl.Chem.; 1993, 30; 865-872.]

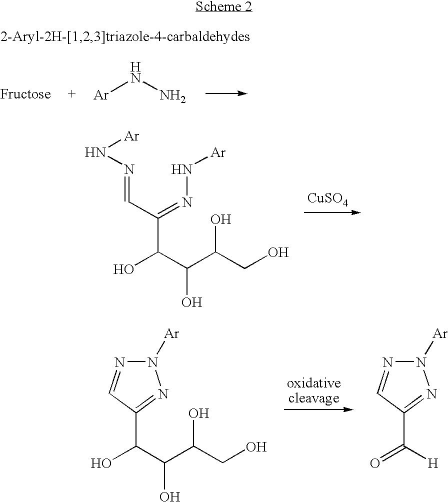

With reference to scheme 2, [1,2,3]triazole-4-carbaldehydes may be obtained from aryl glucosetriazoles by oxidative cleavage, employing for example periodic acid in aqueous mixtures of dioxane or THF at−20 to 120° C. Aryl glucosetriazoles may be obtained by cyclization of the intermediate aryl glucosazone in the presence of copper (II) sulfate in aqueous mixtures of for example dioxane or THF at−20 to 120° C. The aryl glucosazone in turn is made by coupling of arylhydrazines with fructose in acetic acid and water at −20 to 120° C.

[Buckler, R.;Hartzler, H.; Kurchacova, E.; Nichols, G.; Phillips, B.; J. Med. Chem.; 1978; 21(12); 1254-1260, and Riebsomer, J.; Sumrell, G.; J. Org. Chem.; 1948; 13(6); 807-814]

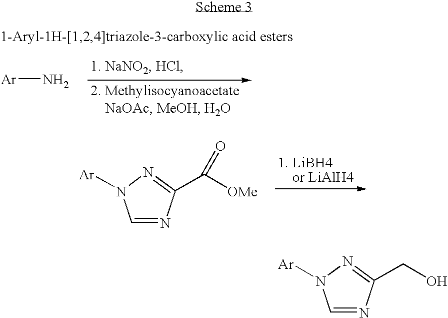

With reference to scheme 3, 1-aryl-1H-1,2,4-triazole-derivatives may be prepared from commercially available anilines by initial diazotization followed by cyclization to the 1,2,4-triazole using methylisocyanocynates (See Matsumoto, K., Suzuki, M., Tomie, M., Yoneda, N. and Miyoshi, M.: Synthesis, 1975, 609-610). The resulting ester is then subjected to reduction to afford the corresponding alcohol (See Genin, M. J. et al: J. Med. Chem. 2000, 43, 953-970).

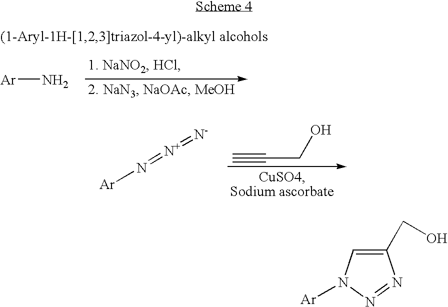

With reference to Scheme 4, 1-aryl-1H-1,2,3-triazole-derivatives may be prepared from commercially available anilines by initial diazotization followed by conversion of the diazonium salt to the corresponding azide using NaN₃. The aryl azide may then be cyclized onto propargyl alcohol in a regiospecific manner using catalytic CuSO₄ to afford the [1,2,3]triazole alcohol intermediate (See Rostovtsev, V. V., Green, L. G., Fokin, V. V., Sharpless, K. B.: Angew., Chem. Intl. Ed. 2002, 41, 14, 2596-2599.)

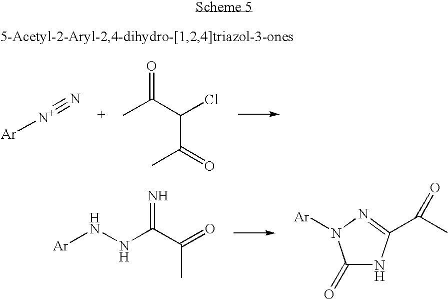

With reference to scheme 5, 5-acetyl-[1,2,4]triazole-3-ones may be made by cyclization of 2-oxo-N′-arylpropanimidohydrazide with carbonyl dichloride or carbonyl diimidazole in solvents such as toluene, dioxane, or THF at temperatures from 40 to 140° C. 2-Oxo-N′-arylpropanimidohydrazides may be synthesized by reaction of aryldiazonium salts, for example the tetrafluoroborate salt, with 3-haloopentane-2,4-diones, for example with halo=chloro, in the presence of potassium acetate in methanol/water at temperatures from −40 to 40° C. to give an intermediate which is subsequently treated in-situ with ammonia in for example methanol, ethanol, dioxane or THF [U.S. Pat. No. 4,400,517, 1983].

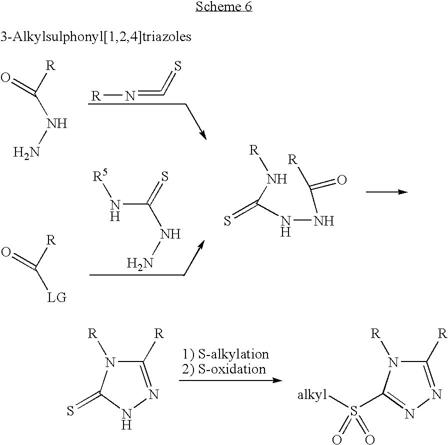

With reference to scheme 6, 3-alkylsulphonyl[1,2,4]triazoles may be prepared from the corresponding dihydro-[1,2,4]triazolethiones by initial alkylation of the sulphur atom with primary alkyl halides such as MeI and EtI (alkyl is Me and Et respectively) in MeOH, EtOH, THF, acetone or the like at −30 to 100° C., followed by oxidation of the sulphur atom using for example KMnO₄ in mixtures of water and acetic acid, or MCPBA in DCM, at −20 to 120° C., or by using any other suitable oxidant. Dihydro[1,2,4]triazolethiones are for example prepared by initial N-acylation of a thiosemicarbazide, using any suitable acylating agent such as acid chlorides, bromides or fluorides (LG is Cl, Br or F) in for example pyridine, or acids (LG is OH), that are activated in situ by the treatment with standard activating reagents such as DCC, DIC, EDCl or HBTU, with or without the presence of co-reagents such as HOBt or DMAP, in suitable solvents such as DMF, DCM, THF, or MeCN at a temperature from −20 to 100° C., followed by ring closure of the initially formed acyclic intermediate either spontaneously under the conditions of the acylation, or by heating at 50 to 150° C. in pyridine or in aqueous solvents in the presence of a base, such as NaHCO₃ or Na₂CO₃, with or without co-solvents such as dioxane, THF, MeOH, EtOH or DMF. This acyclic intermediate can also be formed by treatment of the proper acyl hydrazide with a suitable isothiocyanate in for example 2-propanol, DCM, THF or the like at −20 to 120° C.

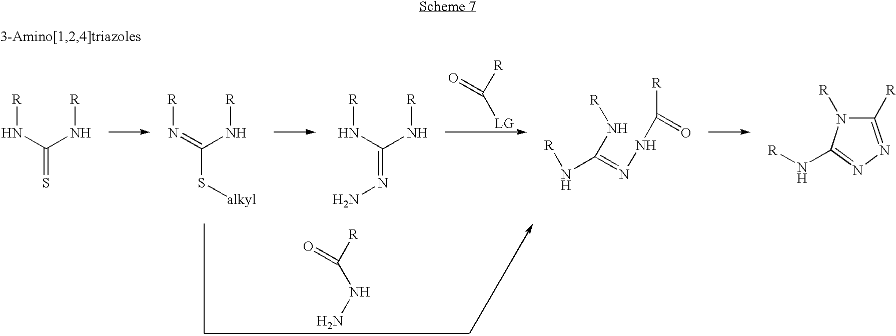

With reference to scheme 7, 3-amino[1,2,4]triazoles may be obtained by treating carbonohydrazonic diamides with a suitable acylating agent carrying a leaving group LG in suitable solvent such as THF, pyridine or DMF at −20 to 100° C. The reaction initially leads to an intermediate that either forms a triazole ring spontaneously, or can be made to do so by heating at 50 to 200° C. in for example pyridine or DMF. The leaving group LG may be chloro or any other suitable leaving group as for example generated by in situ treatment of the corresponding acid (LG is OH) with standard activating reagents as described herein above. Carbonohydrazonic diamides may be generated from isothioureas, in which the S-alkyl (for example S-Me or S-Et) moiety acts as a leaving group upon treatment with hydrazine in solvents such as pyridine, methanol, ethanol, 2-propanol, THF or the like at −20 to 180° C. The intermediate may also be directly generated by treatment of isothioureas with acyl hydrazides under the same conditions as described for the reaction with hydrazine. Isothioureas are obtained by S-alkylation of the corresponding thioureas with for example MeI or EtI in acetone, EtOH, THF, DCM or the like at −100 to 100° C.

Other 5-Membered Heteroaromatics

Other methods for the preparation of 5-membered heteroaromatic rings that are useful for the preparation of compounds of formula I are found in the literature and in books such as “Katritzky and A. F. Pozharskii, Handbook of Heterocyclic Chemistry, Pergamon Press, 2^{nd} Ed. 2000.”

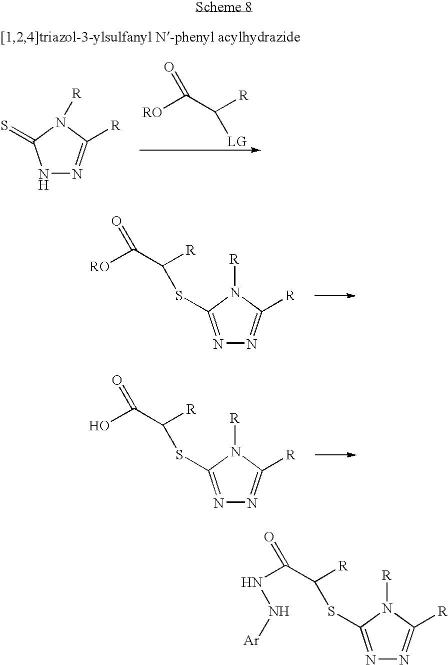

With reference to scheme 8, [1,2,4]triazol-3-ylsulfanyl N′-aryl acylhydrazides may be obtained by reaction of the corresponding acid with aryl hydrazines by standard coupling conditions as described herein above. The acid may be obtained by hydrolysis of its corresponding alkyl ester using standard conditions such as potassium hydroxide in solvents such as methanol or THF/water at temperatures from 0 to 100° C. Alkylation of a triazole thione with for example methyl chloro acetate or propionate under standard conditions as described herein below gives the alkyl ester.

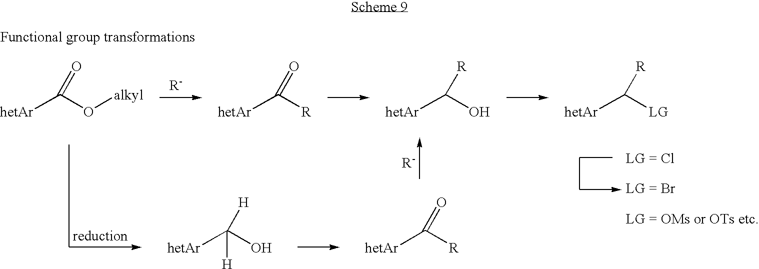

With reference to scheme 9, aliphatic alcohols may for example be converted by standard methods to the corresponding halides by the use of for example triphenylphosphine in combination with either iodine, N-bromosuccinimide or N-chlorosuccinimide, or alternatively by treatment with tribromophosphine or thionyl chloride. Alcohols may be transformed to other leaving groups such as mesylates or tosylates by employing the appropriate sulfonyl halide or sulfonyl anhydride in the presence of a non-nucleophilic base together with the alcohol to obtain the corresponding sulfonates. Chlorides or sulfonates may be converted to the corresponding bromides or iodides by treatment with bromide salts, for example LiBr, or iodide salts, such as LiI. Further standard methods to obtain alcohols include the reduction of the corresponding carbonyl containing groups such as methyl or ethyl esters, aldehydes or ketones, by employing common reducing agents such as boranes, lithium borohydride, lithium aluminium hydride, or hydrogen in the presence of a transition metal catalyst such as complexes of for example ruthenium or iridium, or alternatively palladium on charcoal. Ketones and secondary alcohols may be obtained by treatment of carboxylic acid esters and aldehydes respectively, with the appropriate carbon nucleophile, such as alkyl-Grignard reagents or alkyl-lithium reagents according to standard protocols. Heteroaromatic aldehydes may be prepared from the corresponding primary alcohols by oxidation procedures well known to the one skilled in the art, such as the employment of MnO₂ as oxidant, or by Swern oxidation.
Stereoselective Preparation of Chiral Secondary Alcohols

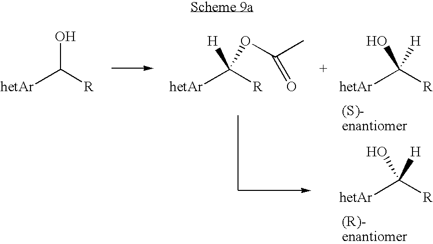

Enantiomerically pure or enriched products, as depicted in scheme 9a, (R is Me or Et) are obtained by kinetic resolution of racemic or scalemic secondary alcohols using enzyme-catalyzed acetylation with for example polymer bound Candida Antarctica Lipase (Novozyme 435®, or other esterases, for example Candida rugosa or Pseudomonas fluorescens, in organic solvents such as toluene, tert-butyl methyl ether tert-butanol or DCM at temperatures from 0 to 90° C., using acetylating reagents such as vinyl acetate, other substituted alkyl acetates, pentafluorophenyl acetate or nitro- or halophenyl acetates, which yields the enriched (R)-acetate and the enriched (S)-alcohol. The (R)-acetate may be hydrolyzed to the corresponding alcohol by e.g. lithium hydroxide in mixtures of THF and water or by any other methods as described herein below, to yield the opposite enantiomerically enriched or pure alcohol.

Preparation of Final Compounds

The subsequent described non-limiting methods of preparation of final compounds of formula I are illustrated and exemplified by drawings in which the generic groups, or other structural elements of the intermediates correspond to those of formula I. It is to be understood that an intermediate containing any other generic group or structural element than those of formula I can be used in the exemplified reactions, provided that this group or element does not hinder the reaction and that it can be chemically converted to the corresponding group or element of formula I at a later stage which is known to the one skilled in the art.

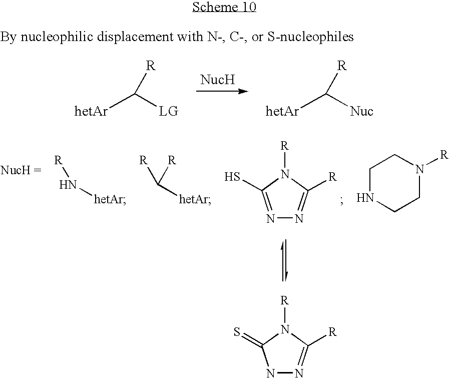

With reference to scheme 10, compounds of formula I may for example be prepared by bond formation through nucleophilic displacement of a leaving group (LG) in which the nucleophilic atom might be the amino-nitrogen atom of a heterocyclic amine, the alpha-carbon of an alkyl substituted heteroaromatic, the sulphur atom of a [1,2,4]triazole-3-thiol tautomer and the nitrogen atom of a secondary aliphatic amine, such as piperazine derivatives. Amino-nitrogen atoms of heterocyclic amines, and the alpha-carbons of alkyl substituted heteroaromatics, are generally not reactive in the neutral protonated form and are therefore preferably fully or partly converted to more nucleophilic anionic forms by treatment with bases in suitable solvents such as lithium diispropylamine or n-BuLi in THF, diethyl ether or toluene, or NaH in for example DMF, or K₂CO₃ or Cs₂CO₃ in acetonitrile or ketones such as 2-butanone, either in situ or just before the reaction with a suitable electrophile carrying a leaving group, at a temperature from −100 to 150° C. The sulphur atoms of [1,2,4]triazole-3-thiols and the nitrogen atoms of secondary aliphatic amines may be nucleophilic enough to displace a leaving group in the corresponding neutral forms, but preferably a base such as K₂CO₃, Cs₂CO₃, TEA, DEA or the like is added to facilitate the reaction in solvents such as acetonitrile, DMF or DCM at 0 to 150° C. For carbon nucleophiles, the leaving group is preferable bromo, for other nucleophiles examples of suitable leaving groups LG include chloro, bromo, OMs and OTs. Optionally, catalytic or stoichiometric amounts of an alkali metal iodide, such as LiI, may be present in the reaction to facilitate the same through in situ displacement of the leaving group to iodo.

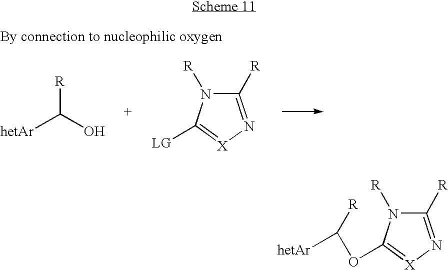

With reference to scheme 11, compounds of formula I (wherein X⁴ as drawn in formula I is O) may be prepared by bond formation through nucleophilic replacement of a leaving group (LG) in which an alcohol acts as O-nucleophile under basic conditions. The base used may include strong hydridic bases, for example NaH or milder bases, such as Cs₂CO₃, at temperatures from 0 to 80° C. in polar aprotic solvents such as DMF or acetonitrile, whereas for chiral alcohols the preferred base is Cs₂CO₃ in order to obtain enantiomerically pure products directly. Examples of suitable leaving groups are alkylsulfonyls such as methanesulfonyl and ethanesulfonyl, and halogens, such as chloro.

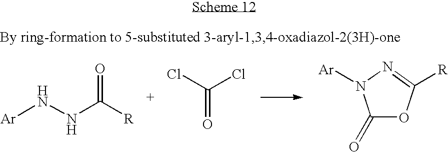

With reference to scheme 12, compounds of formula I may be prepared by condensing suitably substituted acyl hydrazides with phosgene in the presence of bases, such as TEA or DEA, in solvents such as dioxane, THF, DCM, toluene or DMF at 50 to 200° C. as described for similar oxadiazolones in e.g. J. Med. Chem. 1993, 36, 1157-1167.

The invention further relates to the following compounds, which may be used as intermediates in the preparation of compounds of formula I;

Methyl-(4-methyl-5-pyridin-4-yl-4H-[1,2,4]triazol-3-yl)-amine

4-Methyl-5-pyridin-3-yl-2,4-dihydro-3H-1,2,4-triazole-3-thione

4-Methyl-5-pyridin-4-yl-2,4-dihydro-[1,2,4]triazole-3-thione

4-Cyclopropyl-5-pyridin-4-yl-2,4-dihydro-3H-1,2,4-triazole-3-thione

4-(4-Methyl-5-methylsulfanyl-4H-[1,2,4]triazol-3-yl)-pyridine

4-(4-Cyclopropyl-5-methylsulfanyl-4H-[1,2,4]triazol-3-yl)-pyridine

4-(5-Methanesulfonyl-4-methyl-4H-[1,2,4]triazol-3-yl)-pyridine

4-(4-Cyclopropyl-5-methanesulfonyl-4H-[1,2,4]triazol-3-yl)-pyridine

Methyl [(4-methyl-5-pyridin-3-yl-4H-1,2,4-triazol-3-yl)thio]acetate

[(4-Methyl-5-pyridin-3-yl-4H-1,2,4-triazol-3-yl)thio]acetic acid

N′-(3-Chlorophenyl)-2-[(4-methyl-5-pyridin-3-yl-4H-1,2,4-triazol-3-yl)thio]acetohydrazide

5-(1-Chloroethyl)-2-(3-chlorophenyl)-1,2-dihydro-3H-1,2,4-triazol-3-one

2-(3-chlorophenyl)-5-(1-hydroxyethyl)-2,4-dihydro-3H-1,2,4-triazol-3-one

5-acetyl-2-(3-chlorophenyl)-2,4-dihydro-3H-1,2,4-triazol-3-one

N′-(3-chlorophenyl)-2-oxopropanimidohydrazide

Ethyl 1-(3-chlorophenyl)-1H-pyrazole-4-carboxylate

[1-(3-chlorophenyl)-1H-pyrazol-4-yl]methanol

1-(3-chlorophenyl)-1H-pyrazole-4-carbaldehyde

1-[1-(3-chlorophenyl)-1H-pyrazol-4-yl]ethanol

1-[2-(3-chlorophenyl)-2H-1,2,3-triazol-4-yl]ethanol

4-(1-chloroethyl)-2-(3-chlorophenyl)-2H-1,2,3-triazole

1-(3-chlorophenyl)-1H-1,2,4-triazole-3-carboxylic acid methyl ester

[1-(3-Chloro-phenyl)-1H-[1,2,4]triazol-3-yl]-methanol

Methanesulfonic acid 1-(3-chloro-phenyl)-1H-[1,2,4]triazol-3-ylmethyl ester

[1-(3-Chloro-phenyl)-1H-[1,2,3]triazol-4-yl]-methanol

Methanesulfonic acid 1-(3-chloro-phenyl)-1H-[1,2,3]triazol-4-ylmethyl ester

### EXAMPLES

The invention will now be illustrated by the following non-limiting examples.

General Methods

All starting materials are commercially available or earlier described in the literature.

The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, m: multiplet).

Analytical in line liquid chromatography separations followed by mass spectra detections, were recorded on a Waters LCMS consisting of an Alliance 2795 (LC) and a ZQ single quadropole mass spectrometer. The mass spectrometer was equipped with an electrospray ion source operated in a positivé and/or negative ion mode. The ion spray voltage was ±3 kV and the mass spectrometer was scanned from m/z 100-700 at a scan time of 0.8 s. To the column, X-Terra MS, Waters, C8, 2.1×50 mm, 3.5 mm, was applied a linear gradient from 5% to 100% acetonitrile in 10 mM ammonium acetate (aq.), or in 0.1% TFA (aq.). Preparative reversed phase chromatography was run on a Gilson autopreparative HPLC i 5 with a diode array detector using an XTerra MS C8, 19×300 mm, 7 mm as column. Purification by a chromatotron was performed on rotating silica gel/gypsum (Merck, 60 PF-254 with calcium sulphate) coated glass sheets, with coating layer of 1, 2, or 4 mm using a TC Research 7924T chromatotron. Purification of products were also done by flash chromatography in silica-filled glass columns.

Microwave heating was performed in a Smith Synthesizer Single-mode microwave cavity producing continuous irradiation at 2450 MHz (Personal Chemistry AB, Uppsala, Sweden).

### Example 1

### Methyl-(4-methyl-5-pyridin-4-yl-4H-[1,2,4]triazol-3-yl)-amine

A mixture of 1000 mg (4.35 mmol) N-amino-N′,N″-dimethyl-guanidine hydriodide (Henry; Smith; J.Amer.Chem.Soc.; 73; 1951; 1858) and 774 mg (4.35 mmol) isonicotinoyl chloride hydrochloride in 3 ml of pyridine was heated under microwave irradiation for 5 min at 160° C. Aq. sat. K₂CO₃ was added and the mixture was extracted with CHCl₃. The combined organic layer was dried and concentrated. Recrystallization from ethanol, water and EA gave 216 mg (26%) of the title compound. ¹H NMR (d6-DMSO): 2.85 (d, 3 H) 3.45 (s, 3 H) 6.25 (d, 1 H) 7.65 (m, 2 H) 8.67 (m, 2 H)

### Example 2

### 4-Methyl-5-pyridin-3-yl-2,4-dihydro-3H-1,2,4-triazole-3-thione

A solution of 4-methyl-3-thiosemicarbazide (902 mg, 8.58 mmol), nicotinic acid (960 mg, 7.80), EDCl (1.64 g, 8.58 mmol), HOBt (1.16 g, 8.58 mmol) in DMF (10 mL) was stirred at r.t. o.n. The reaction mixture was diluted with EA (100 mL), successively washed with 10% aq. hydrochloric acid, water, sat. aq. Na₂CO₃, water and then brine. The organic phase was dried (Na₂SO₄), filtered and concentrated in vacuo. The residue was stirred in NaOH (53.4 mL, 66.7 mmol, 5% aq.) at 60° C. o.n. The mixture was cooled to r.t., then brought to pH about 6 using 1N aq. HCl. The aq. phase was sat. with solid NaCl, then extracted with EA. The combined organic phase was washed with brine, dried (Na₂SO₄), filtered, concentrated and dried in vacuo to give the title compound (180 mg). ¹H-NMR: 11.6 (br s, 1H), 8.94 (s, 1H), 8.83 (dd, 1H), 7.98 (m, 1H), 7.51 (dd, 1H), 3.69 (s, 3H).

### Example 3

### 4-Methyl-5-pyridin-4-yl-2,4-dihydro-[1,2,4]triazole-3-thione

Isonicotinoyl chloride hydrochloride (27.5 g, 154.5 mmol) and 4-methyl-3-thiosemicarbazide (16.4 g, 155.9 mmol) were mixed in pyridine (200 ml) and stirred under argon at ambient temperature overnight. After evaporation to dryness, aqueous sodium hydroxide (250 mL, 2M, 500 mmol) was added and the resulting solution was heated at 60° C. for 16 h. After cooling to room temperature, the solution was neutralized with 6N hydrochloric acid. The precipitate that formed was collected by filtration to give the title compound (pale yellow solid, 16.4 g, 55%). ¹H NMR (DMSO-d6), δ (ppm): 8.78 (dd, 2H), 7.75 (dd, 2H), 3.59 (s, 3H).

### Example 4

### 4-Cyclopropyl-5-pyridin-4-yl-2,4-dihydro-3H-1,2,4-triazole-3-thione

Isonicotinohydrazide (5.4 g, 39 mmol) and cyclopropyl isothiocyanate (4.1 g, 41 mmol) were mixed in 2-propanol (100 ml) and heated to 70° C. o.n. The reaction was cooled to r.t. and evaporated to dryness. H₂O (170 mL) followed by NaHCO₃ (6.7 g, 80 mmol) was added to the residue and the mixture was refluxed o.n. The reaction mixture was cooled to rt, acidified with concentrated hydrochloric acid and the title compound 9.0 g (94%) was collected by filtration. ¹H NMR: 0.63 (m, 2 H) 1.00 (m, 2 H) 3.25 (m, 1 H) 7.75 (d, 2 H) 8.74 (m, 2 H)

### Example 5

### 4-(4-Methyl-5-methylsulfanyl-4H-[1,2,4]triazol-3-yl)-pyridine

To a solution of 4-Methyl-5-pyridin-4-yl-2,4-dihydro-[1,2,4]triazole-3-thione (1000 mg, 5.20 mmol) in 1M sodium hydroxide (10 mL), added a solution of iodomethane (0.52 mL, 8.32 mmol) in ethanol (3 mL). Stirred at RT overnight. Extracted into 200 mL dichloromethane and washed with brine (50 mL). Dried over anhydrous sodium sulfate, filtered and concentrated in vacuo to yield title compound (1.00 g, 94% yield). ¹H-NMR (CDCl₃) δ (ppm): 8.81 (d, 2H), 7.62 (d, 2H), 3.68 (s, 3H), 2.82 (s, 3H).

### Example 6

### 4-(4-Cyclopropyl-5-methylsulfanyl-4H-[1,2,4]triazol-3-yl)-pyridine

A solution of iodomethane (0.457 mL, 7.33 mmol) in ethanol (3 mL) was added to a solution of 4-cyclopropyl-5-pyridin-4-yl-2,4-dihydro-3H-1,2,4-triazole-3-thione (1 g, 4.58 mmol) in 1M sodium hydroxide (10 mL) at room temperature. After stirring overnight, the reaction mixture was extracted with dichloromethane and then the organic layer was washed with brine, dried over anhydrous sodium sulfate, filtered and concentrated to afford the titled compound (729.1 mg, 69%, beige solid). ¹H NMR (CDCl₃) δ (ppm): 8.77 (d, 2H), 7.75 (m, 2H), 3.23 (m, 1H), 2.82 (s, 3H), 1.17 (m, 2H), 0.80 (m, 2H).

### Example 7

### 4-(5-Methanesulfonyl-4-methyl-4H-[1,2,4]triazol-3-yl)-pyridine

To a solution of 4-(4-methyl-5-methylsulfanyl-4H-[1,2,4]triazol-3-yl)-pyridine (1000 mg, 4.85 mmol) in acetic acid, added a solution of KMnO₄ (1.15 g, 7.28 mmol) in H₂O (50 mL) drop-wise. Stirred at RT for 3 hours. Added sodium hydrogen sulfite until purple color was discharged. Extracted into chloroform (3×100 mL). Washed organic layer with saturated sodium bicarbonate (50 mL). Dried over anhydrous sodium sulfate, filtered and concentrated in vacuo to yield title compound (1.01 g, 87% yield). ¹H-NMR (CDCl₃) δ (ppm): 8.89 (d, 2H), 7.64 (d, 2H), 4.05 (s, 3H), 3.64 (s, 3H).

### Example 8

### 4-(4-Cyclopropyl-5-methanesulfonyl-4H-[1,2,4]triazol-3-yl)-pyridine

A solution of potassium permanganate (525 mg, 3.3 mmol) in water (22.0 mL) was added to a solution of 4-(4-cyclopropyl-5-methylsulfanyl-4H-[1,2,4]triazol-3-yl)-pyridine (514 mg, 2.2 mmol) in acetic acid (11 mL) drop-wise at room temperature. After stirring for 3 hours, sodium hydrogen sulfite was added until the purple color was discharged. The reaction mixture was extracted with chloroform and then the organic layer was washed with saturated sodium bicarbonate, dried over anhydrous sodium sulfate, filtered and concentrated to afford the titled compound (546.7 mg, 94%, white solid). ¹H NMR (CDCl₃) δ (ppm): 8.86 (d, 2H), 7.77 (d, 2H), 3.64 (m, 1H), 3.63 (s, 3H), 1.25 (m, 2H), 1.01 (m, 2H).

### Example 9

### Methyl [(4-methyl-5-pyridin-3-yl-4H-1,2,4-triazol-3-yl)thio]acetate

1.75 g (9.15 mmol) 4-Methyl-5-pyridin-3-yl-2,4-dihydro-3H-1,2,4-triazole-3-thione and 2.47 g (17.8 mmol) K₂CO₃ were dissolved, respectively suspended in MeCN (25 mL) and five drops of DMF were added, followed by 0.81 mL (9.18 mmol) methyl chloroacetate. The reaction was stirred under argon at r.t. o.n. After filtration the filtrate was taken up in EA and washed with water. To the aq. layer was added brine and sodium bicarbonate, followed by extraction with DCM and EA. All organic layers were pooled and evaporated to dryness. Flash chromatography (DCM/MeOH=70/1 to 10/1) gave 2.19 g (91%) of the title compound. ¹H-NMR: 8.89 (d, 1H), 8.74 (dd, 1H), 8.01 (dd, 1H), 7.46 (m, 1H), 4.11 (s, 2H), 3.77 (s, 3H), 3.70 (s, 3H)

### Example 10

### [(4-Methyl-5-pyridin-3-yl-4H-1,2,4-triazol-3-yl)thio]acetic acid

2.00 g (7.50 mmol) methyl [(4-methyl-5-pyridin-3-yl-4H-1,2,4-triazol-3-yl)thio]acetate was dissolved in MeOH (30 mL). 0.45 g (8.0 mmol) Potassium hydroxide was added. After stirring at r.t. for 18 h the temperature was increased to 50° C. After further 3 h more potassium hydroxide was added (0.20 g) and stirring continued for additional 3 h. The mixture was cooled, diluted with aq. KOH and washed with EA. The aq. layer was acidified to pH 2 and evaporated to dryness, giving crude title product, which was used directly in the next step. ¹H-NMR(DMSO-d6): 8.98 (d, 1 H), 8.80 (dd, 1 H), 8.26-8.35 (m, 1 H), 7.73 (dd, 1 H), 4.07 (s, 2 H), 3.66 (s, 3 H).

### Example 11

### N′-(3-Chlorophenyl)-2-[(4-methyl-5-pyridin-3-yl-4H-1,2,4-triazol-3-yl)thio]acetohydrazide

Crude [(4-methyl-5-pyridin-3-yl-4H-1,2,4-triazol-3-yl)thio]acetic acid from the previous step was dissolved under argon in DMF/MeCN (20 mL/20 mL), followed by addition of 1.04 g (7.81 mmol) HOBt, 1.40 g (7.30 mmol) EDCl, 2 mL (20.6 mmol) DEA and 0.85 g (7.86 mmol) 3-chlorophenylhydrazine. After stirring for 1.5 hours the volume was reduced in vacuo and diluted with water. Extraction with EA, followed by washing with Na₂CO₃, citric acid and finally brine gave after evaporation a crude which was purified over silica (DCM/MeOH=30/1) yielding 1.07 g (40%) of the title compound. ¹H-NMR (DMSO-D6): 8.89-8.93 (m, 1 H), 8.74 (dd, 1 H), 8.07-8.18 (m, 2 H), 7.60 (dd, 1 H), 7.09 (t, 1 H), 6.62-6.74 (m, 3 H), 4.03 (s, 2 H), 3.65 (s, 4 H).

### Example 12

### 5-(1-Chloroethyl)-2-(3-chlorophenyl)-1,2-dihydro-3H-1,2,4-triazol-3-one

SOCl₂ (1 mL, 8.4 mmol) was added to a solution of 2-(3-chlorophenyl)-5-(1-hydroxyethyl)-2,4-dihydro-3H-1,2,4-triazol-3-one (500 mg, 2.1 mmol) in DCM (15 mL). After stirring for 3 h the solvent and excess SOCl₂ were removed in-vacuo. Flash chromatography (MeOH/DCM 1:30) gave the title compound in 500 mg yield. ¹H NMR: 1.9 (d, 3 H) 5.0 (q, 1 H) 7.2 (ddd, 1 H) 7.4 (t, 1 H) 7.9 (dt, 1 H) 8.0 (t, 1 H) 11.9 (s, 1 H)

### Example 13

### 2-(3-chlorophenyl)-5-(1-hydroxyethyl)-2,4-dihydro-3H-1,2,4-triazol-3-one

Sodium borohydride (300 mg, 7.9 mmol) in water (70 mL) was added to a solution of 5-acetyl-2-(3-chlorophenyl)-2,4-dihydro-3H-1,2,4-triazol-3-one (2 g, 8.4 mmol) in MeOH (40 mL). Acetic acid (2 mL) was added after stirring for 5 min. The MeOH was removed under reduced pressure. After 12 h at 7° C. the title compound was filtered off as in 2 g yield. ¹H-NMR: 1.5 (d, 3 H) 4.7 (q, 1 H) 7.1 (d, 1 H) 7.3 (m, 1 H) 7.8 (d, 1 H) 7.9 (s, 1 H)

### Example 14

### 5-acetyl-2-(3-chlorophenyl)-2,4-dihydro-3H-1,2,4-triazol-3-one

Phosgene (3.5 mmol) in toluene (1.8 mL) was added dropwise to a mixture of N′-(3-chlorophenyl)-2-oxopropanimidohydrazide (500 mg, 2.7 mmol) and pyridine (560 μl,7.1 mmol) in toluene (5 mL). The mixture was stirred for 2 h at r.t. under nitrogen, followed by filtration and washing with toluene. The solid was taken up in DCM and washed with water and brine. The solution was dried and concentrated. Flash chromatography (MeOH/DCM 1:40) gave the title compound in 200 mg yield. LC-MS (M⁺-1) 236

### Example 15

### N′-(3-chlorophenyl)-2-oxopropanimidohydrazide

3-Chlorobenzenediazonium tetrafluoroborate (10 g, 44 mmol) in water (300 mL) was added to a mixture of 3-chloropentane-2,4-dione (6 g, 44 mmol) and potassium acetate (8 g, 88 mmol) in MeOH (500 mL) at 0° C. After stirring for 30 min a formed solid was filtered off and recrystallized from MeOH. The crystals were re-dissolved in MeOH (200 mL) and the solution was added to 7 M ammonia in MeOH (100 mL). After stirring for 1 h, water was added leading to a precipitate which was filtered off and dried, giving the title compound in 5.5 g yield. LC-MS (M⁺-1) 210

### Example 16

### Ethyl 1-(3-chlorophenyl)-1H-pyrazole-4-carboxylate

3-Chlorophenylhydrazine hydrochloride (4.6 g, 25.7 mmol) in EtOH (100 mL) was added at 0° C. to a stirred solution of ethyl 2-formyl-3-oxopropanoate (3.7 g, 25.7 mmol) [J.Heterocyclic Chem. 1993, 30, 865-872] in EtOH (80 mL). After addition was completed the reaction was allowed to reach rt, followed by stirring o.n. The reaction mixture was concentrated and the residue was recrystallized from EtOH to give 4.2 g (65%) of the title compound. ¹H NMR: 1.29 (t, 3H) 4.25 (q, 2H) 7.25 (d, 1H) 7.34 (t, 1H) 7.51 (d, 1H) 7.68 (s, 1H) 8.01 (s, 1H) 8.37 (s, 1H)

### Example 17

### [1-(3-chlorophenyl)-1H-pyrazol-4-yl]methanol

A solution of ethyl 1-(3-chlorophenyl)-1H-pyrazole-4-carboxylate (4.2 g, 16.8 mmol) in Et₂O (100 mL) was slowly added to a stirred solution of LiAlH₄ (1.65 g, 43 mmol) in Et₂O (80 mL) at rt under nitrogen. The mixture was allowed to reach rt and was stirred for additional 1.5 h, followed by quenching via sequential addition of H₂O (2.6 mL), THF (6 mL) and 15% aq. NaOH (2.6 mL). The mixture was stirred for 20 min, dried with Na₂SO₄, filtered and evaporated to dryness to give 3.4 g (97%) of the title compound. ¹H NMR: 4.68 (s, 2H) 7.24 (m, 1H) 7.36 (t, 1H) 7.53 (m, 1H) 7.72 (m, 2H) 7.91 (s, 1H)

### Example 18

### 1-(3-chlorophenyl)-1H-pyrazole-4-carbaldehyde

MnO₂ was added to a solution of [1-(3-chlorophenyl)-1H-pyrazol-4-yl]methanol (3.4 g) in DCM (60 mL) at rt. The mixture was stirred at 40° C. o.n. The mixture was filtered through celite and the celite was washed with DCM (100 mL). The filtrate was evaporated to dryness to give 2.5 g (76%) of the title compound. ¹H NMR: 7.35 (d, 1H) 7.33 (t, 1H) 7.60 (d, 1H) 7.79 (t, 1H) 8.16 (s, 1H) 8.43 (s, 1H) 9.96 (s, 1H)

### Example 19

### 1-[1-(3-chlorophenyl)-1H-pyrazol-4-yl]ethanol

A solution of 1-(3-chlorophenyl)-1H-pyrazole-4-carbaldehyde (2.5 g, 12 mmol) in Et₂O (100 mL) was added to MeMgCl in THF (11 mL, 3 M, 30 mmol) at 0° C. The reaction was stirred at 0° C. for 15 min and at rt for 2 h. Sat. aq. NH₄Cl was added and the mixture was extracted with Et₂O. The organic phase was dried and concentrated to give 2.7 g (100 %) of the title compound. ¹H NMR: 1.50 (d, 3H) 4.92 (q, 1H) 7.18 (m, 1H) 7.30 (t, 1H) 7.49 (m, 1H) 7.63 (s, 1H) 7.66 (t, 1H) 7.81 (s, 1H)

### Example 20

### 1-[2-(3-chlorophenyl)-2H-1,2,3-triazol-4-yl]ethanol

A solution of 2-(3-chlorophenyl)-2H-1,2,3-triazole-4-carbaldehyde (1.2 g, 5.8 mmol) [J.Med. Chem, 1978, 21, 1254-1260] in Et₂O (70 mL) was added to MeMgCl in THF (4.8 mL, 3 M, 14.4 mmol) at 0° C. The reaction was stirred at 0° C. for 30 min and at rt for 1 h. Sat. aq. NH₄Cl was added and the mixture was extracted with EA. The organic phase was dried and concentrated to give 1.14 g (100%) of the title compound. ¹H NMR: 1.58 (d, 3H) 5.08 (q, 1H) 7.25 (m, 1H) 7.33 (t, 1H) 7.71 (s, 1H) 7.88 (m, 1H) 8.02 (t, 1H)

### Example 21

### 4-(1-chloroethyl)-2-(3-chlorophenyl)-2H-1,2,3-triazole

2 drops of DMF were added to 1-[2-(3-chlorophenyl)-2H-1,2,3-triazol-4-yl]ethanol (190 mg, 0.85 mmol) in SOCl₂ (3 mL) and the reaction was heated at 70° C. for 2 h. The excess SOCl₂ was evaporated and the residue was dried in vacuo to give the title compound in 206 mg (100%) yield. ¹H NMR: 1.95 (d, 3H) 5.28 (q, 1H) 7.31 (m, 1H) 7.40 (t, 1H) 7.83 (s, 1H) 7.95 (m, 1H) 8.08 (t, 1H)

### Example 22

### 1-(3-chlorophenyl)-1H-1,2,4-triazole-3-carboxylic acid methyl ester

A solution of 3-chlorobenzenediazonium chloride was prepared from 3-chloroaniline (2.2 mL, 21 mmol) in 10% HCl (35 mL) and sodium nitrite (1.73 g, 25 mmol) in water (8 mL) 0° C. This solution was added drop-wise with stirring to a mixture of methyl isocyanate (1.8 mL, 20 mmol), sodium acetate (13.1 g, 160 mmol), methanol (80 mL) and water (24 mL) over a period of 30 minutes at 0-5° C. Stirring was continued for 1 h at the same temperature; then, methanol was removed in vacuo and the resultant products were extracted with EtOAc (500 mL). The combined organics were washed successively with 1 N HCl (100 mL), saturated NaHCO₃ (100 mL), water (100 mL) and brine (50 mL), then dried (Na₂SO₄), filtered and concentrated. The crude solid was recrystallized from boiling benzene to give 1.54 g (32%) of the title compound as a brown solid. ¹H NMR (CDCl₃) δ (ppm): 8.66 (s, 1H), 7.84 (m, 1H), 7.66 (m, 1 H), 7.47-7.53 (m, 2H), 4.08 (s, 3H), 1.60 (s, 2H).

### Example 23

### [1-(3-Chloro-phenyl)-1H-[1,2,4]triazol-3-yl]-methanol

A mixture of lithium borohydride (94 mg, 4.3 mmol) in 2-propanol (17 mL) was treated with 1-(3-chlorophenyl)-1H-1,2,4-triazole-3-carboxylic acid methyl ester (0.50 g, 2.1 mmol). The flask was closed, and the reaction stirred overnight at room temperature. Water (5 mL) was added to decompose excess hydride, and the reaction mixture was adsorbed onto silica gel. Chromatography (SPE, 60-100% EtOAc in hexanes) gave 186 mg (42%) of the desired product as a white solid. ¹H NMR (CDCl₃) δ (ppm): 8.55 (s 1H), 7.75 (t, 1H), 7.58 (dt, 1H), 7.47 (t, 1 H), 7.40 (dt, 1H), 4.88 (d, 2H), 2.41 (t, 1H).

### Example 24

### Methanesulfonic acid 1-(3-chloro-phenyl)-1H-[1,2,4]triazol-3-ylmethyl ester

[1-(3-Chloro-phenyl)-1H-[1,2,4]triazol-3-yl]-methanol (87 mg, 0.42 mmol) was suspended in CH₂Cl₂ (5 mL) and the suspension was cooled to 0° C. To this was added methanesulfonyl chloride (0.050 mL, 0.65 mmol) and triethylamine (0.12 mL, 0.86 mol). This solution was stirred at 0° C. for 1 h. To the reaction mixture in an ice bath was added cold saturated NaHCO₃ solution (5 mL). The organic phase was washed with brine (5 mL) then dried (Na₂SO₄), filtered and concentrated under educed pressure to give 99 mg (78%) of a yellow oil, which NMR showed to be a 1:2 mixture of the title compound and 3-Chloromethyl-1-(3-chloro-phenyl)-1H-[1,2,4]triazole. ¹H NMR (CDCl₃) δ (ppm): 8.59 (s, 0.67 H), 8.55 (s, 0.33 H), 7.71 (t, 1 H), 7.58 (dt, 1H), 7.41-7.49 (m, 2H), 5.42 (s, 1.27 H), 4.73 (s, 0.79 H), 2.82 (s, 2.3 H).

### Example 25

### [1-(3-Chloro-phenyl)-1H-[1,2,3]triazol-4-yl]-methanol

1-Azido-3-chlorobenzene (0.56 g, 3.7 mmol) and propargyl alcohol (0.18 mL, 3.1 mmol) were dissolved in t-butanol/water 1:1 (12 mL). Sodium ascorbate (1 M solution, 0.6 mL, 0.6 mmol) and copper sulfate pentahydrate (15 mg, 0.06 mmol) were added, and the mixture was stirred at room temperature for 16 h. The mixture was diluted with EtOAc and washed with water and brine, dried (Na₂SO₄), and concentrated. Chromatography (SPE, 5% MeOH in 1:1 EtOAc/CH₂Cl₂) gave 275 mg (42 %) of the title compound as a white solid. ¹H NMR (CDCl₃) δ (ppm): 8.00 (d, J=0.5 Hz, 1H), 7.80 (apparent t, J=2 Hz, 1H), 7.65 (dq, J=8, 2 Hz, 1H), 7.45-7.49 (m, 2H), 4.92 (d, J=7 Hz, 2H), 2.48 (t, J=7 Hz, 1H).

### Example 26

### Methanesulfonic acid 1-(3-chloro-phenyl)-1H-[1,2,3]triazol-4-ylmethyl ester

Methanesulfonyl chloride (0.11 mL, 1.4 mmol) was added to a solution of [1-(3-chlorophenyl)-1H-[1,2,3]triazol-4-yl]-methanol (0.20 g, 0.95 mmol) and triethylamine (0.27 mL, 1.9 mmol) in CH₂Cl₂ (10 mL) at 0° C., and the mixture was stirred at 0° C. for 1.5 h. Cold NaHCO₃ (saturated solution, 5 mL) was added, then the organic phase was washed with brine, dried (Na₂SO₄), filtered and concentrated crude yellow oil was triturated with ether is to give 0.17 g (63%) of the title compound as a white solid. ¹H NMR (CDCl₃) δ (ppm): 8.18 (s, 1H), 7.82 (td, 1H), 7.67 (dt, 1H), 7.45-7.55 (m, 2H), 5.48 (d, 2H), 5.48 (d, 2H).

### Example 27

### 3-(3-chlorophenyl)-5-{[(4-methyl-5-pyridin-3-yl-4H-1,2,4-triazol-3-yl)thio]methyl}-1,3,4-oxadiazol-2(3H)-one

1.04 g (2.77 mmol) N′-(3-chlorophenyl)-2-[(4-methyl-5-pyridin-3-yl-4H-1,2,4-triazol-3-yl)thio]acetohydrazide was suspended in THF (100 mL) and cooled on an ice-water bath. 0.45 mL (5.62 mmol) TEA and 0.51 mg (3.14 mmol) CDI were added and the reaction was stirred under Ar at r.t. for 15.5 hours. Since no conversion had taken place, dioxane (50 mL) was added giving a homogeneous reaction mixture which was heated to 68° C. To this, additional 0.45 mL (5.62 mmol) TEA and 0.51 mg (3.14 mmol) CDI were added and finally 1.5 mL (2.8 mmol) of 20% phosgene in toluene together with 0.45 mL (5.62 mmol) TEA, followed by stirring for 2 h. Additional same amounts of phosgene and TEA were added after this and stirring again for 30 minutes. The reaction mixture was reduced in vacuo to about ⅔ of original volume, poured on ice/brine and extracted with EA, followed by washing with Na₂CO₃. The aq. layers were re-extracted with EA and the organics pooled, dried (Na₂SO₄) and evaporated to dryness. The crude was filtered over silica (DCM/MeOH=30/1) and purified over silica using DCM/MeOH=30/1, giving crude product which was further purified over silica using a slow gradient DCM neat to DCM/MeOH=80/1 to 1/1 giving after evaporation and drying 593 mg (53%) of the title compound. ¹H NMR: 8.87 (s, 1 H), 8.72 (d, 1 H), 7.93-8.06 (m, 1 H), 7.77 (t, 1 H), 7.64-7.73 (m, 1 H), 7.44 (dd, 1 H), 7.30 (t, 1 H), 7.15-7.21 (m, 1 H), 4.45 (s, 2 H), 3.67 (s, 3 H)

### Example 28

### 2-(3-chlorophenyl)-5-{1-[methyl(4-methyl-5-pyridin-4-yl-4H-1,2,4-triazol-3-yl)amino]ethyl}-2,4-dihydro-3H-1,2,4-triazol-3-one

NaH (3 mg, 0.1 mmol) was added to a solution of methyl-(4-methyl-5-pyridin-4-yl-4H-[1,2,4]triazol-3-yl)-amine (16 mg, 0.09 mmol) in DMF(2 mL) under nitrogen. After stirring for 10 min a solution of 5-(1-chloroethyl)-2-(3-chlorophenyl)-1,2-dihydro-3H-1,2,4-triazol-3-one (20 mg, 0.08 mmol) in DMF (1 mL) was added. After stirring for 1 h, 10 mL sat. aq. ammonium chloride was added and the mixture was extracted with EA. The organic phase was dried and concentrated. Prep. HPLC gave the desired product in 9 mg yield. ¹H NMR: 1.6 (d, 3 H), 2.8 (s, 3 H), 3.6 (s, 3 H), 4.7 (q, 1 H), 7.2 (d, 1 H), 7.3 (t, 1 H), 7.6 (s, 2 H), 8.0 (d, 1 H), 8.0 (s, 1 H), 8.8 (s, 2 H), 11.4 (s, 1 H)

### Example 29

4-(5-{1-[1-(3-chlorophenyl)-1H-pyrazol-4-yl]ethoxy}-4-methyl-4H-1,2,4-triazol-3-yl)pyridine

NaH (28 mg, 1.16 mmol) was added to a solution of 1-[1-(3-chlorophenyl)-1H-pyrazol-4-yl]ethanol (100 mg, 0.45 mmol) and 4-[4-methyl-5-(methylsulfonyl)-4H-1,2,4-triazol-3-yl]pyridine (101 mg, 0.45 mmol) in DMF (5 mL). The reaction was stirred at 60° C. o.n. Brine was added and the mixture was extracted with EA. The organic phase was dried and concentrated. The product was purified by flash column chromatography (DCM to DCM-MeOH 40:1) afforded 43 mg (25%) of the title compound. ¹H NMR: 1.79 (d, 3H), 3.42 (s, 3H), 5.61 (q, 1H), 7.21 (m, 1H), 7.32 (t, 1H), 7.52 (m, 3H), 7.67 (t, 1H), 7.73 (s, 1H), 7.93 (s, 1H), 8.73 (d, 2H)

### Example 30

### 4-(5-{1-[2-(3-chlorophenyl)-2H-1,2,3-triazol-4-yl]ethoxy}-4-methyl-4H-1,2,4-triazol-3-yl)pyridine

Cs₂CO₃ (171 mg, 0.52 mmol) was added to a solution of 3-[4-methyl-5-(methylsulfonyl)-4H-1,2,4-triazol-3-yl]pyridine (80 mg, 0.35 mmol) and 1-[2-(3-chlorophenyl)-2H-1,2,3-triazol-4-yl]ethanol (80 mg, 0.35 mmol) in DMF (20 mL). The reaction was stirred at 60° C. for 40 h. Brine was added and the mixture was extracted with EA. The organic phase was dried and concentrated. The product was purified by flash column chromatography (DCM to DCM-MeOH 40:1) to afford 23 mg (17%) of the title compound. ¹H NMR: 1.95 (d, 3H), 3.57 (s, 3H), 6.40 (q, 1H), 7.32 (d, 1H), 7.39 (t, 1H), 7.60 (m, 2H), 7.95 (m, 2H), 8.09 (t, 1H), 8.74 (d, 2H)

### Example 31

### 4-[5-({1-[2-(3-chlorophenyl)-2H-1,2,3-triazol-4-yl]ethyl}thio)-4-cyclopropyl-4H-1,2,4-triazol-3-yl]pyridine

Cs₂CO₃ (130 mg, 0.40 mmol) was added to a solution of 4-cyclopropyl-5-pyridin-4-yl-2,4-dihydro-3H-1,2,4-triazole-3-thione (85 mg, 0.39 mmol) and 4-(1-chloroethyl)-2-(3-chlorophenyl)-2H-1,2,3-triazole (95 mg, 0.39 mmol) in DMF (4 mL). The reaction was stirred at 60° C. for 24 h. Brine was added and the mixture was extracted with EA. The organic phase was dried and concentrated. The product was purified by flash column chromatography (DCM to DCM-MeOH 40:1) to afford 113 mg (68%) of the title compound. ¹H NMR: 0.73 (m, 2H), 1.12 (m, 2H), 1.98 (d, 3H), 3.16 (m, 1H), 5.42 (q, 1H), 7.30 (m, 1H), 7.40 (t, 1H), 7.70 (dd, 2H), 7.86 (s, 1H), 7.94 (m, 1H), 8.07 (t, 1H), 8.75 (dd, 2H)

### Example 32

### 4-{5-[1-(3-Chloro-phenyl)-1H-[1,2,4]triazol-3-ylmethylsulfanyl]-4-cyclopropyl-4H-[1,2,4]triazol-3-yl}-pyridine

A solution of methanesulfonic acid 1-(3-chloro-phenyl)-1H-[1,2,4]triazol-3-ylmethyl ester (28 mg, 0.09 mmol), potassium carbonate (38 mg, 0.27 mmol) and 4-cyclopropyl-5-pyridin-4-yl-2,4-dihydro-[1,2,4]triazole-3-thione (20 mg, 0.09 mmol) in acetonitrile (3 mL) was stirred at room temperature for 16 h. The reaction mixture was diluted with EtOAc (15 mL), then washed with water (10 mL). The aqueous phase was re-extracted with CH₂Cl₂ (10 mL), and the combined organics were washed with brine (15 mL), dried (Na₂SO₄), filtered and concentrated onto silica gel. Flash chromatography (SPE, 2-5% MeOH in 1:1 CH₂Cl₂/EtOAc) gave 14 mg (38%) of the title compound as a white solid. ¹H NMR (CDCl₃) δ (ppm): 8.78 (d, J=6 Hz, 2 H), 8.53 (s, 1H), 7.73-7.78 (m, 3H), 7.57 (dt, J=8, 2 Hz, 1 H), 7.46 (t, J=8 Hz, 1 H), 7.39 (dt, J=8, 2 Hz, 1 H), 4.82 (s, 2H), 3.29 (5, J=4 Hz, 1H), 1.15-1.28 (m, 4H).

### Example 33

### 4-{5-[1-(3-Chloro-phenyl)-1H-[1,2,4]triazol-3-ylmethoxy]-4-cyclopropyl-4H-[1,2,4]triazol-3-yl}-pyridine

Sodium hydride (60% oil dispersion, 12 mg, 0.30 mmol) was added to a solution of [1-(3-chloro-phenyl)-1H-[1,2,4]triazol-3-yl]-methanol (47 mg, 0.22 mmol) in DMF (3 mL) under argon and the mixture was stirred for 45 minutes. 4-(5-Methanesulfonyl-4-methyl-4H-[1,2,4]triazol-3-yl)-pyridine (39 mg, 0.15 mmol) was added, and the mixture was heated to 80° C. and stirred for 40 h. The reaction mixture was extracted with EtOAc (50 mL) and CH₂Cl₂ (25 mL), and the combined organics were washed with water (3×20 mL) and brine (30 mL), then dried (Na₂SO₄), filtered and concentrated onto silica gel. Chromatography (SPE, 5% MeOH in 1:1 CH₂Cl₂/EtOAc) afforded 18 mg of the title compound as a white solid. ¹H NMR (CDCl₃) δ (ppm): 8.75 (d, 2H), 8.60 (d, 1H), 7.78-7.80 (m, 3H), 7.78 (m, 1H), 7.67-7.74 (m, 3H), 5.76 (d, 2H), 3.22 (m, 1H), 1.08-1.12 (m, 2H), 0.86-0.90 (m, 2H).

### Example 34

### 4-{5-[1-(3-Chloro-phenyl)-1H-[1,2,3]triazol-4-ylmethylsulfanyl]-4-methyl-4H-[1,2,4]triazol-3-yl}-pyridine

A mixture of methanesulfonic acid 1-(3-chloro-phenyl)-1H-[1,2,3]triazol-4-ylmethyl ester (40 mg, 0.14 mmol), potassium carbonate (58 mg, 0.42 mmol) and 4-methyl-5-pyridin-4-yl-2,4-dihydro-[1,2,4]triazole-3-thione (27 mg, 0.14 mmol) in acetonitrile (5 mL) was stirred at room temperature for 18 h. The reaction mixture was diluted with EtOAc and washed with water, and the aqueous phase was re-extracted with CH₂Cl₂. The combined organics were washed with water and brine, then dried (Na₂SO₄), filtered and concentrated onto silica gel. Chromatography (SPE, 5-15% MeOH in 1:1 CH₂Cl₂/EtOAc) yielded 39 mg (73%) of a white solid. ¹H NMR (CDCl₃) δ (ppm): 8.80 (dd, J=5, 2 Hz, 2H), 8.26 (s, 1H), 7.78 (t, J=2 Hz, 1H), 7.58-7.64 (m, 3H), 7.46 (t, J=7 Hz, 1H), 7.42 (dt, J=7, 2 Hz, 1H), 4.71 (s, 2H), 3.65 (s 3H).

### Example 35

### 4-{5-[1-(3-Chloro-phenyl)-1H-[1,2,3]triazol-4-ylmethylsulfanyl]-4-cyclopropyl-4H-[1,2,4]triazol-3-yl}-pyridine

A mixture of methanesulfonic acid 1-(3-chloro-phenyl)-1H-[1,2,3]triazol-4-ylmethyl ester (40 mg, 0.14 mmol), potassium carbonate (58 mg, 0.42 mmol) and 4-cyclopropyl-5-pyridin-4-yl-2,4-dihydro-[1,2,4]triazole-3-thione (31 mg, 0.14 mmol) in acetonitrile (5 mL) was stirred at room temperature for 18 h. The reaction mixture was diluted with EtOAc and washed with water, and the aqueous phase was re-extracted with CH₂Cl₂. The combined organics were washed with water and brine, then dried (Na₂SO₄), filtered and concentrated onto silica gel. Chromatography (SPE, 5-15% MeOH in 1:1 CH₂Cl₂EtOAc) yielded 45 mg (79%) of a white solid. ¹H NMR (CDCl₃) δ (ppm): 8.78 (dd, 2H), 8.32 (s, 1H), 7.79 (t, 1 H), 7.74 (dd, 2H), 7.63 (dt, 1H), 7.37-7.48 (m, 2H), 4.74 (s, 2H), 3.23 (m, 1H), 1.14-1.27 (m, 2H), 0.77-0.82 (m, 2H).

### Example 36

### 4-{5-[1-(3-Chloro-phenyl)-1H-[1,2,3]triazol-4-ylmethoxy]-4-cyclopropyl-4H-[1,2,4]triazol-3-yl}-pyridine

Sodium hydride (60% oil dispersion, 13 mg, 0.32 mmol) was added to a solution of [1-(3-chloro-phenyl)-1H-[1,2,3]triazol-4-yl]-methanol (50 mg, 0.24 mmol) in DMF (3 mL), and the mixture was stirred for 45 minutes at room temperature. 4-(4-Cyclopropyl-5-methanesulfonyl-4H-[1,2,4]triazol-3-yl)-pyridine (42 mg, 0.16 mmol) was added, and the mixture was heated to 80° C. in an oil bath and stirred for 40 h. The mixture was diluted with EtOAc (30 mL) and washed with water (2×15 mL), and the aqueous phases were combined and re-extracted with CH₂Cl₂ (10 mL). The combined organics were washed with brine (2×10 mL), dried (Na₂SO₄), filtered and concentrated onto silica gel. Chromatography (SPE, 3-30% MeOH in 1:1 CH₂Cl₂/EtOAc) afforded 18 mg (19%) of the title compound as a white solid. ¹H NMR (CDCl₃) δ (ppm): 9.75 (m, 2H), 8.44 (s, 1H), 7.83 (td, J=2, 0.5 Hz, 1H), 7.76 (dd, J=5, 2 Hz, 2H), 7.67 (dt, J=7, 2 Hz, 1H), 7.42-7.51 (m, 2H), 5.77 (s, 2H), 3.16 (7, J=4 Hz, 1H), 1.08-1.16 (m, 2H), 0.76-0.80 (m, 2H).

### Example 37

### (1R)-1-[2-(3-chlorophenyl)-2H-1,2,3-triazol-4-yl]ethyl acetate and (1S)-1-[2-(3-chlorophenyl)-2H-1,2,3-triazol-4-yl]ethanol

Vinyl acetate (350 μL, 3.8 mmol) was added to 1-[2-(3-chlorophenyl)-2H-1,2,3-triazol-4-yl]ethanol (650 mg, 2.9 mmol) and Novozyme 435® (80 mg) in toluene (10 mL) and the mixture was stirred at r.t. under an argon-atmosphere for 24 h. The mixture was filtered through celite and the celite was washed with DCM. The combined filtrate was evaporated and the residue was purified by flash column chromatography (SiO₂, DCM to DCM-MeOH 40:1) to give 320 mg (45%) of (1R)-1-[2-(3-chlorophenyl)-2H-1,2,3-triazol-4-yl]ethyl acetate. ¹H NMR: 1.70 (d, 3 H), 2.12 (s, 3 H), 6.13 (q, 1 H), 7.33 (m, 1 H), 7.41 (t, 1 H), 7.77 (s, 1 H), 7.97 (dd, 1 H), 8.10 (t, 1 H), (1S)-1-[2-(3-chlorophenyl)-2H-1,2,3-triazol-4-yl]ethanol was also obtained, in 49% yield. ¹H NMR: 1.65 (d, 3 H), 5.15 (q, 1 H), 7.30 (m, 1 H), 7.40 (t, 1 H), 7.78 (s, 1 H), 7.95 (m, 1 H), 8.10 (t, 1 H)

### Example 38

### (1R)-1-[2-(3-chlorophenyl)-2H-1,2,3-triazol-4-yl]ethanol

Lithium hydroxide monohydrate (102 mg, 2.43 mmol) was added to (1R)-1-[2-(3-chlorophenyl)-2H-1,2,3-triazol-4-yl]ethyl acetate (323 mg, 1.21) in THF/water 1:1 (10 mL). After 18 h stirring at r.t the volume of the mixture was reduced in vacuo to about ½, followed by dilution with brine and extraction with EtOAc, 270 mg (100%) of the title compound was obtained after evaporation and drying. ¹H NMR: 1.64 (d, 3 H), 5.13 (q, 1 H), 7.31 (m, 1 H), 7.39 (t, 1 H), 7.76 (s, 1 H), 7.94 (m, 1 H), 8.08 (t, 1 H)

### Example 39

### 4-(5-{(1R)-[2-(3-chlorophenyl)-2H-1,2,3-triazol-4-yl]ethoxy}-4-methyl-4H-1,2,4-triazol-3-yl)pyridine

Cs₂CO₃ (326 mg, 1.0 mmol) was added to a solution (1R)-1-[2-(3-chlorophenyl)-2H-1.2.3-triazol-4-yl]ethanol (149 mg, 0.67 mmol) and 4-[4-methyl-5-(methylsulfonyl)-4H-1,2,4-triazol-3-yl]pyridine (149 mg, 0.66 mmol) in DMF (5 mL). The reaction was stirred at 60° C. for 48 h. Brine was added and the mixture was extracted 3 times with EtOAc. The organic phase was dried and concentrated. The product was purified by flash column chromatography (SiO₂, DCM to DCM-MeOH 40: 1) to give 69 mg (27%) of the title compound. ¹H NMR: 1.95 (d, 3 H), 3.57 (s, 3 H), 6.40 (q, 1 H), 7.32 (m, 1 H), 7.40 (t, 1 H), 7.65 (d, 2 H), 7.97 (m, 2 H), 8.10 (t, 1 H), 8.76 (br. s., 2 H)

Pharmacology

The pharmacological properties of the compounds of the invention can be analyzed using standard assays for functional activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay that measures the mobilization of intracellular calcium, [Ca²⁺]ᵢ in cells expressing mGluR5.

For FLIPR analysis, cells expressing human mGluR5d as described in WO97/05252 were seeded on collagen coated clear bottom 96-well plates with black sides and analysis of [Ca²⁺]ᵢ mobilization was done 24 h after seeding.

FLIPR experiments were done using a laser setting of 0.800 W and a 0.4 second CCD camera shutter speed. Each FLIPR experiment was initiated with 160 μl of buffer present in each well of the cell plate. After each addition of the compound, the fluorescence signal was sampled 50 times at 1 second intervals followed by 3 samples at 5 second intervals. Responses were measured as the peak height of the response within the sample period. EC₅₀ and IC₅₀ determinations were made from data obtained from 8-point concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

We have validated a secondary functional assay for mGluR5d as described in WO97/05252 based on Inositol Phosphate (IP₃) turnover. IP₃ accumulation is measured as an index of receptor mediated phospholipase C turnover. GHEK cells stably expressing the human mGluR5d receptors were incubated with [3H] myo-inositol overnight, washed three times in HEPES buffered saline and pre-incubated for 10 min with 10 mM LiCl. Compounds (agonists) were added and incubated for 30 min at 37° C. Antagonist activity was determined by pre-incubating test compounds for 15 min, then incubating in the presence of glutamate (80 μM) or DHPG (30 μM) for 30 min. Reactions were terminated by the addition of perchloric acid (5%). Samples were collected and neutralized, and inositol phosphates were separated using Gravity-Fed Ion-Exchange Columns.

A detailed protocol for testing the compounds of the invention is provided in the assay below.

Assay of Group I Receptor Antagonist Activity

For FLIPR analysis, cells expressing human mGluR5d as described in WO97/05252 were seeded on collagen coated clear bottom 96-well plates with black sides and analysis of [Ca²⁺]ᵢ mobilization was performed 24 h following seeding. Cell cultures in the 96-well plates were loaded with a 4 μM solution of acetoxymethyl ester form of the fluorescent calcium indicator fluo-3 (Molecular Probes, Eugene, Oreg.) in 0.01% pluronic. All assays were performed in a buffer containing 127 mM NaCl, 5 mM KCl, 2 mM MgCl₂, 0.7 mM NaH₂PO₄, 2 mM CaCl₂, 0.422 mg/ml NaHCO₃, 2.4 mg/ml HEPES, 1.8 mg/ml glucose and 1 mg/ml BSA Fraction IV (pH 7.4).

FLIPR experiments were done using a laser setting of 0.800 W and a 0.4 second CCD camera shutter speed with excitation and emission wavelengths of 488 nm and 562 nm, respectively. Each FLIPR experiment was initiated with 160 μl of buffer present in each well of the cell plate. A 40 μl addition from the antagonist plate was followed by a 50 μL addition from the agonist plate. After each addition the fluorescence signal was sampled 50 times at 1 second intervals followed by 3 samples at 5 second intervals. Responses were measured as the peak height of the response within the sample period.

EC₅₀/IC₅₀ determinations were made from data obtained from 8 points concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

Measurement of Inositol Phosphate Turnover in Intact Whole Cells

GHEK stably expressing the human mGluR5d receptor were seeded onto 24 well poly-L-lysine coated plates at 40×10⁴ cells /well in media containing 1 μCi/well [3H] myo-inositol. Cells were incubated overnight (16 h), then washed three times and incubated for 1 h at 37° C. in HEPES buffered saline (146 mM NaCl, 4.2 mM KCl, 0.5 mM MgCl₂, 0.1% glucose, 20 mM HEPES, pH 7.4) supplemented with 1 unit/ml glutamate pyruvate transaminase and 2 mM pyruvate. Cells were washed once in HEPES buffered saline and pre-incubated for 10 min in HEPES buffered saline containing 10 mM LiCl. Compounds (agonists) were added and incubated at 37° C. for 30 min. Antagonist activity was determined by pre-incubating test compounds for 15 min, then incubating in the presence of glutamate (80 μM) or DHPG (30 μM) for 30 min. The reaction was terminated by the addition of 0.5 ml perchloric acid (5%) on ice, with incubation at 4° C. for at least 30 min. Samples were collected in 15 ml Falcon tubes and inositol phosphates were separated using Dowex columns, as described below.

Assay For Inositol Phosphates Using Gravity-Fed Ion-Exchange Columns

Preparation of Ion-Exchange Columns

Ion-exchange resin (Dowex AG1-X8 formate form, 200-400 mesh, BIORAD) was washed three times with distilled water and stored at 4° C. 1.6 ml resin was added to each column, and washed with 3 ml 2.5 mM HEPES, 0.5 mM EDTA, pH 7.4.

a) Sample Treatment

Samples were collected in 15 ml Falcon tubes and neutralized with 0.375 M HEPES, 0.75 M KOH. 4 ml of HEPES/EDTA (2.5/0.5 mM, pH 7.4) were added to precipitate the potassium perchlorate. Supernatant was added to the prepared Dowex columns.

b) Inositol Phosphate Separation

Elute glycero phosphatidyl inositols with 8 ml 30 mM ammonium formate.

Elute total inositol phosphates with 8 ml 700 mM ammonium formate/100 mM formic acid and collect eluate in scintillation vials. Count eluate mixed with 8 ml scintillant.

One aspect of the invention relates to a method for inhibiting activation of mGluR5, comprising treating a cell containing said receptor with an effective amount of the compound of formula I.

Screening for Compounds Active Against tlesr

Adult Labrador retrievers of both genders, trained to stand in a Pavlov sling, are used. Mucosa-to-skin esophagostomies are formed and the dogs are allowed to recover completely before any experiments are done.

Motility Measurement

In brief, after fasting for approximately 17 h with free supply of water, a multilumen sleeve/sidehole assembly (Dentsleeve, Adelaide, South Australia) is introduced through the esophagostomy to measure gastric, lower esophageal sphincter (LES) and esophageal pressures. The assembly is perfused with water using a low-compliance manometric perfusion pump (Dentsleeve, Adelaide, South Australia). An air-perfused tube is passed in the oral direction to measure swallows, and an antimony electrode monitored pH, 3 cm above the LES. All signals are amplified and acquired on a personal computer at 10 Hz.

When a baseline measurement free from fasting gastric/LES phase III motor activity has been obtained, placebo (0.9% NaCl) or test compound is administered intravenously (i.v., 0.5 ml/kg) in a foreleg vein. Ten min after i.v. administration, a nutrient meal (10% peptone, 5% D-glucose, 5% Intralipid, pH 3.0) is infused into the stomach through the central lumen of the assembly at 100 ml/min to a final volume of 30 ml/kg. The infusion of the nutrient meal is followed by air infusion at a rate of 500 ml/min until an intragastric pressure of 10±1 mmHg is obtained. The pressure is then maintained at this level throughout the experiment using the infusion pump for further air infusion or for venting air from the stomach. The experimental time from start of nutrient infusion to end of air insufflation is 45 min. The procedure has been validated as a reliable means of triggering TLESRs. TLESRs is defined as a decrease in lower esophageal sphincter pressure (with reference to intragastric pressure) at a rate of >1 mmHg/s. The relaxation should not be preceded by a pharyngeal signal ≦2s before its onset in which case the relaxation is classified as swallow-induced. The pressure difference between the LES and the stomach should be less than 2 mmHg, and the duration of the complete relaxation longer than 1 s.

Abbreviations

BSA Bovine Serum Albumin

CCD Charge Coupled Device

CRC Concentration Response Curve

DHPG 3,5-dihydroxyphenylglycine;

EDTA Ethylene Diamine Tetraacetic Acid

FLIPR Fluorometric Imaging Plate reader

GHEK GLAST-containing Human Embrionic Kidney

GLAST glutamate/aspartate transporter

HEPES 4-(2-hydroxyethyl)-1-piperazineethanesulfonic acid (buffer)

IP₃ inositol triphosphate

Results

Typical IC₅₀ values as measured in the assays described above are 10 μM or less. In one aspect of the invention the IC₅₀ is below 2 μM. In another aspect of the invention the IC₅₀ is below 0.2 μM. In a further aspect of the invention the IC₅₀ is below 0.05 μM.

| Compound | FLIPR IC₅₀ | |
|---|---|---|
| 4-(5-{1-[2-(3-chlorophenyl)-2H-1,2,3- | 27 | nM |
| triazol-4-yl]ethoxy}-4-methyl- | | |
| 4H-1,2,4-triazol-3-yl)pyridine | | |
| 4-[5-({[1-(3-chlorophenyl)-1H- | 265 | nM |
| 1,2,3-triazol-4-yl]methyl}thio)-4- | | |
| cyclopropyl-4H-1,2,4-triazol-3-yl]pyridine | | |

1. A compound according to Formula II, 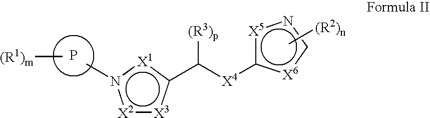
wherein,
P is aryl;
if m=1 then R¹ is attached to P at the meta position of the ring P relative to the attachment point of P to the 5-membered ring, and if m=2 then R¹ is attached to P at the 2-, and 5-positions of the ring P to the 5-membered ring;
R¹ is selected from the group consisting of hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶,C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing atoms independently selected from the group consisting of C, N, O and S;
R⁵ and R⁶ are independently selected from a group consisting of hydrogen, C₁₋₆alkyl, C₃₋₇cycloalkyl and aryl;
X¹ and X² are independently selected from the group consisting of CR⁴, and N;
X³ is selected from the group consisting of CR⁴, N, and O; wherein at least one of X¹ X² and X³ is not N;
R⁴ is selected from the group consisting of H, ═O, C₁₋₆alkyl, OH;
R³ is selected from the group consisting of H, C₁₋₆alkyl, hydroxy, C₀₋₆alkylcyano, oxo, ═NR⁵, ═NOR⁵, C₁₋₄alkylhalo, halo, C3-7cycloalkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, OC₁₋₄alkyl, C₁₋₄alkylOR⁵ and C₀₋₄alkylNR⁵R⁶;
X⁴ is selected from the group consisting of CR⁷R⁸, NR⁷, O, S, SO, and SO₂;
R⁷ and R⁸ are independently selected from a group consisting of hydrogen, C₁₋₆alkyl, C₃₋₇cycloalkyl and aryl;
X⁵ and X⁶ are independently selected from the group consisting of C, N, O and S;
R² is selected from the group consisting of hydroxy, C₀₋₆alkylcyano, ═NR⁵, ═NOR⁵, C₁₋₄alkylhalo, halo, C₁₋₆alkyl, C₃₋₆cycloalkyl, C₀₋₆alkylaryl, C_{0-,6}alkylheteroaryl, C₀₋₆alkylcycloalkyl, C₀₋₆alkylheterocycloalkyl, OC₁₋₄alkyl, OC₀₋₆alkylaryl, O(CO)C₁₋₄alkyl, (CO)OC₁₋₄alkyl, C₀₋₄alkyl(S)C₀₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₁₋₄alkylOR⁵, C₀₋₄alkylNR⁵R⁶ and a 5- or 6-membered ring containing atoms independently selected from C, N, O and S, and wherein said ring may be substituted by one or more A; and
any C₁₋₆alkyl, aryl or heteroaryl defined under R¹, R² and R³ may be substituted by one or more A;
A is selected from the group consisting of hydrogen, hydroxy, halo, nitro, oxo, C₀₋₆alkylcyano, C₀₋₄alkylC₃₋₆cycloalkyl, C₁₋₆alkyl, C₁₋₆alkylhalo, OC₁₋₆alkylhalo, C₂₋₆alkenyl, C₀₋₃alkylaryl, C₀₋₆alkylOR⁵, OC₂₋₆alkylOR⁵, C₁₋₆alkylSR⁵, OC₂₋₆alkylSR⁵, (CO)R⁵, O(CO)R⁵, OC₂₋₆alkylcyano, OC₁₋₆alkylCO₂R⁵, O(CO)OR⁵, OC₁₋₆alkyl(CO)R⁵, C₁₋₆alkyl(CO)R⁵, NR⁵OR⁶, C₁₋₆alkylNR⁵R⁶, OC₂₋₆alkylNR⁵R⁶, C₀₋₆alkyl(CO)NR⁵R⁶, OC₁₋₆alkyl(CO)NR⁵R⁶, OC₂₋₆alkylNR⁵(CO)R⁶, C₀₋₆alkylNR⁵(CO)R⁶, C₀₋₆alkylNR⁵(CO)NR⁵R⁶, O(CO)NR⁵R⁶, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, SO₃R⁵, C₁₋₆alkylNR⁵(SO₂)NR⁵R⁶, OC₂₋₆alkyl(SO₂)R⁵, C₀₋₆alkyl(SO₂)R⁵, C₀₋₆alkyl(SO)R⁵, OC₂₋₆alkyl(SO)R⁵ and a 5- or 6-membered ring containing one or more atoms independently selected from the group consisting of C, N, O and S;
m is selected from 1 and 2;
n is selected from 0, 1, 2, 3 and 4;
p is selected from 1 and 2; and
and a salts or hydrates thereof,

2. A compound according to claim 1 wherein P is phenyl.

3. A compound according to claim 1 wherein X⁴ is selected from CR⁷R⁸, NR⁷, O and S.

4. A compound according to claim 1 wherein X⁵ is N.

5. A compound according to claim 4 wherein X⁶ is N.

6. A compound according to claim 4 wherein X⁶ is O.

7. A compounds according to claim 1 wherein X⁵ is C and X⁶ is N.

8. A compound according to claim 1 wherein R² is selected from aryl and C₀₋₆heteroaryl

9. A compound according to claim 1 wherein R² is selected from 4-pyridyl, 3-pyridyl and phenyl.

10. A compound according to claim 1 wherein R² is a 5- or 6-membered ring containing atoms independently selected from C, N, O and S, which ring may be substituted by one or more A.

11. A compound according to claim 1 wherein the ring containing X¹, X², and X³ is selected from the group consisting of:

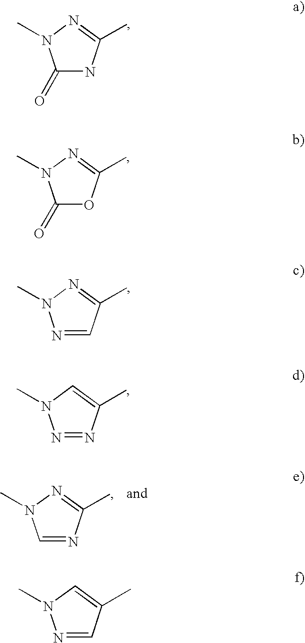

12. A compound according to claim 1 wherein X¹ and X² are N and X³ is C.

13. A compound according to claim 1 selected from the group consisting of:
3-(3-chlorophenyl)-5-{[(4-methyl-5-pyridin-3-yl-4H-1,2,4-triazol-3-yl)thio]methyl}-1,3,4-oxadiazol-2(3H)-one
2-(3-chlorophenyl)-5-{1-[methyl(4-methyl-5-pyridin-4-yl-4H-1,2,4-triazol-3-yl)amino]ethyl}-2,4-dihydro-3H-1,2,4-triazol-3-one
4-(5-{1-[1-(3-chlorophenyl)-1H-pyrazol-4-yl]ethoxy}-4-methyl-4H-1,2,4-triazol-3-yl)pyridine
4-(5-{1-[2-(3-chlorophenyl)-2H-1,2,3-triazol-4-yl]ethoxy}-4-methyl-4H-1,2,4-triazol-3-yl)pyridine
4-[5-({1-[2-(3-chlorophenyl)-2H-1,2,3-triazol-4-yl]ethyl}thio)-4-cyclopropyl-4H-1,2,4-triazol-3-yl]pyridine
4-{5-[1-(3-Chloro-phenyl)-1H-[1,2,4]triazol-3-ylmethylsulfanyl]-4-cyclopropyl-4H-[1,2,4]triazol-3-yl}-pyridine
4-{5-[1-(3-Chloro-phenyl)-1H-[1,2,4]triazol-3-ylmethoxy]-4-cyclopropyl-4H-[1,2,4]triazol-3-yl}-pyridine
4-{5-[1-(3-Chloro-phenyl)-1H-[1,2,3]triazol-4-ylmethylsulfanyl]-4-methyl-4H-[1,2,4]triazol-3-yl}-pyridine
4-{5-[1-(3-Chloro-phenyl)-1H-[1,2,3]triazol-4-ylmethylsulfanyl]-4-cyclopropyl-4H-[1,2,4]triazol-3-yl}-pyridine
4-{5-[1-(3-Chloro-phenyl)-1H-[1,2,3]triazol-4-ylmethoxy]-4-cyclopropyl-4H-[1,2,4]triazol-3-yl}-pyridine, and
4-(5-{(1R)-[2-(3-chlorophenyl)-2H-1,2,3-triazol-4-yl]ethoxy}-4-methyl-4H-1,2,4-triazol-3-yl)pyridine

14. A pharmaceutical composition comprising as active ingredient a therapeutically effective amount of the compound according to claim 1, in association with one or more pharmaceutically acceptable diluent, excipients and/or inert carrier.

15. The pharmaceutical composition according to claim 14, for use in the treatment of mGluR 5 mediated disorders.

16. The compound according to claim 1, for use in therapy.

17. The compound according to claim 1, for use in treatment of mGluR 5 mediated disorders.

18. Use of the compound according to claim 1, in the manufacture of a medicament for the treatment of mGluR 5 mediated disorders.

19. A method of treatment of mGluR 5 mediated disorders, comprising administrering to a mammal, including man in need of such treatment, a therapeutically effective amount of the compound according to claim 1.

20. The method according to claim 19, for use in treatment of neurological disorders.

21. The method according to claim 19, for use in treatment of psychiatric disorders.

22. The method according to claim 19, for use in treatment of chronic and acute pain disorders.

23. The method according to claim 19, for use in treatment of gastrointestinal disorders.

24. A method for inhibiting activation of mGluR 5 receptors, comprising treating a cell containing said receptor with an effective amount of the compound according to claim 1.
